(19) United States
(12) Patent Application Publication
(10) Pub. No.: US 2019/0264112 A1
(43) Pub. Date: Aug. 29, 2019

(54) PROCESS FOR THE MANUFACTURE OF BASE OIL

(71) Applicant: Novvi LLC, Emeryville, CA (US)

(72) Inventors: Shakeel TIRMIZI, Matawan, NJ (US); Eduardo BARALT, Houston, TX (US); Liwenny HO, Oakland, CA (US); Jason Charles ROSALLO, Oakland, CA (US); Jason WELLS, Fremont, CA (US); Wui Sum Willbe HO, Oakland, CA (US)

(21) Appl. No.: 16/411,384

(22) Filed: May 14, 2019

Related U.S. Application Data
(63) Continuation of application No. 15/572,212, filed on Nov. 7, 2017, now abandoned.
(60) Provisional application No. 62/159,153, filed on May 8, 2015.

(51) Int. Cl. C10G 50/02; C07C 11/02; C10G 69/12; C07C 1/24; C07C 5/03; C07C 9/16; B01D 3/14; C10M 105/04; C10M 107/10; B01D 3/12; C10M 177/00

(52) U.S. Cl. CPC C10G 50/02; C10N 2220/10; C10G 69/126; C07C 1/24; C07C 5/03; C07C 9/16; B01D 3/14; C10M 105/04; C10M 107/10; B01D 3/12; C10M 177/00; C10N 2270/00; C10N 2220/028; C10N 2220/022; C10G 2300/1088; C10M 2205/0285; C10G 2400/22; C10G 2300/302; C07C 11/02

(57) ABSTRACT

Processes are provided for producing hydrocarbon base oils from alcohols, including by converting one or more alcohols into linear alpha olefins, and then forming branched oligomers with one or more olefin feedstock(s) which are subsequently hydrogenated and fractionated. A process for the preparation of a mixture of branched saturated hydrocarbons can include (a) forming an oligomerization reaction mixture having an oligomerization catalyst system and an olefin monomer mixture, wherein the olefin monomer mixture has an average carbon number in the range of 9.5 to 13, and at least 10% of the olefin monomers in the olefin monomer mixture have a carbon number difference of at least four carbons, (b) oligomerizing the olefin monomers in the oligomerization reaction mixture to produce an oligomer product, (c) separating unreacted olefin monomer from the oligomer product to produce a purified oligomer product, (d) hydrogenating the purified oligomer product, and (e) distilling the hydrogenated oligomer product.

This application is a continuation application of U.S. application Ser. No. 15/572,212 filed Nov. 7, 2017, which is a U.S. National Phase application of PCT/US2016/031274 filed May 6, 2016, which claims priority to U.S. Application No. 62/159,153 filed May 8, 2015. The disclosures of which are incorporated herein by reference.

The present disclosure is generally directed to the field of lubricants, more specifically to hydrocarbon base oils obtained by the oligomerization of one or more olefin feedstocks. In one embodiment, the olefin feedstock comprises a population of olefins derived from alcohols. In another embodiment, the process comprises the preparation of an olefin feedstock including those manufactured by the dehydration of alcohols, an oligomerization step, a hydrogenation step, and a fractional distillation step.

Base oils are the major constituent in lubricants for automobiles, such as 2-stroke, 4-stroke, gear oil, and transmission oils; aviation, such as turbine; and industrial uses, such as hydraulic fluid, compressor oil, lubricating greases, and process oils. Lubricants typically consist of 60-100% base stock by weight and the remainder in additives to control their fluid properties and improve low temperature behavior, oxidative stability, corrosion protection, demulsibility and water rejection, friction coefficients, lubricities, wear protection, air release, color, and other properties.

The American Petroleum Institute (API) publication API 1509, “Engine Oil Licensing and Certification System, 17th Edition”, defines a base oil or base stock as: “ . . . a lubricant component that is produced by a single manufacturer to the same specifications (independent of feed source or manufacturer's location); that meets the same manufacturer's specification; and that is identified by a unique formula, product identification number, or both. Base stocks may be manufactured using a variety of different processes including but not limited to distillation, solvent refining, hydrogen processing, oligomerization, esterification, and rerefining. Rerefined stock shall be substantially free from materials introduced through manufacturing, contamination, or previous use.” Base oil is the base stock or blend of base stocks used in API-licensed oil.

Generally lubricating base oils are base oils having kinematic viscosity of about 2 mm²/s or greater at 100° C. (KV100, kinematic viscosity measured at 100° C.); a pour point (PP) of about −15° C. or less; and a viscosity index (VI) of 120 or greater.

The oils in Group III are very high viscosity index (VHVI) base oils, which are manufactured from crude oil by hydrocracking and catalytic dewaxing or solvent dewaxing. Group III base oils can also be manufactured by catalytic dewaxing of slack waxes originating from crude oil refining, or by catalytic dewaxing of waxes originating from Fischer-Tropsch synthesis from natural gas or coal based raw materials.

TABLE 1
API Base Oil Classification

| | Viscosity | | | |
|---|---|---|---|---|
| | Index | | | |
| API | (ASTM | | | |
| Group | D2270) | Saturates | Sulphur % | Description |
| I | 80-120 | <90% | >.03% | Conventional (solvents) |
| II | 80-120 | ≥90% | ≤03% | Hydroprocessing Required |
| III | >120 | ≥90% | ≤03% | Severe Hydroprocessing |
| IV | | | | PolyAlphaOlefins (PAO) |
| V | | | | All other basestocks not |
| | | | | included above e.g. esters |

Group IV base oils are polyalphaolefin (PAO, or poly-α-olefin) base oils. PAOs are synthetic hydrocarbon base oils which have good flow properties at low temperatures, relatively high thermal and oxidative stability, low evaporation losses at high temperatures, higher viscosity index, good friction and wear behavior, good hydrolytic stability, and excellent thermal conductivity. PAOs are not toxic and are miscible with mineral oils and esters. Consequently, PAOs are suited for use in engine oils, compressor oils, hydraulic oils, gear oils, and greases. Typically PAO is produced by catalytic oligomerization of alpha olefins ranging from 1-octene to 1-dodecene, with 1-decene being a preferred material, most commonly used as synthetic base oils in modern engine lubricants. PAOs useful as synthetic base oils may be synthesized by homogeneous Friedel-Crafts catalyst such as boron trifluoride (BF₃) or aluminum chloride (AlCl₃), typically followed by hydrogenation to remove residual unsaturation and improve thermo-oxidation stability.

PAOs may be produced by the use of Friedel-Craft catalysts, such as aluminum trichloride or boron trifluoride, and a protic promoter. The alpha olefins generally used as feedstock are those in the C8 to C20 range, most preferably 1-octene, 1-nonene, 1-decene, 1-dodecene, and 1-tetradecene.

Alternatives to the Friedel-Craft process include metallocene catalyst systems. Most of the metallocene-based focus has been on high viscosity index PAOs (HVI-PAOs) and higher viscosity oils for industrial and commercial applications. Examples include U.S. Pat. No. 6,706,828, which discloses a process for producing PAOs from metallocene catalysts with methylalumoxane (MAO). Others have made various PAOs, such as polydecene, using various metallocene catalysts not typically known to produce polymers or oligomers with any specific tacticity. Examples include WO 96/23751, EP 0 613 873, U.S. Pat. Nos. 5,688,887, 6,043,401, WO 03/020856 (equivalent to US 2003/0055184), U.S. Pat. Nos. 5,087,788, 6,414,090, 6,414,091, 4,704,491, 6,133,209, and 6,713,438. Although most of the research on metallocene-based PAOs has focused on higher viscosity oils, recent research has looked at producing low viscosity PAOs for automotive applications. US 2007/0043248 discloses a process using a metallocene catalyst for the production of low viscosity (4 to 10 cSt) PAO basestocks. This technology is attractive because the metallocene-based low viscosity PAO has excellent lubricant properties.

A number of US patents have also used BF₃ to oligomerize linear olefins other than alpha olefins to produce Group V synthetic hydrocarbons having properties similar to group IV PAO base oils. For example, U.S. Pat. No. 4,910,355 describes a process using a mixture of C8-18 olefins, preferably C10 olefins, containing about 50-90 weight percent α-olefins and about 10-50 weight percent internal olefins, and contacting this mixture with a catalytic amount of a Friedel-Crafts catalyst, preferably BF₃, and a catalyst promoter, preferably alcohol or water, at a temperature of about 10°-80° C., washing to remove catalyst, distilling to remove monomer and optionally dimer, and hydrogenating to obtain a substantially saturated olefin oligomer. The resultant oligomer exhibits a pour point that is lower than the pour point obtained with a comparative α-olefin under the same oligomerization conditions.

Large quantities of PAOs are used in a variety of lubricating applications. However, PAOs existing in the market today are derived from fossil fuels, and hence are not renewable.

There is a continuing need for improved base oils, for example, base oils that have a wide operational temperature range, and a continuing need for base oils derived from renewable feedstock.

The present invention relates to a process for production of saturated olefin oligomers for use as a synthetic hydrocarbon base oil by:
a) Preparing a suitable C8-C16 olefin feedstock from the dehydration of alcohols; and
b) Reacting said olefin feedstock with one or more linear olefins to form oligomers.

A further object of the invention is an alternative process for the manufacture of branched, saturated hydrocarbons suitable for Group IV PAO base oils.

The process according to the invention comprises multiple steps where, in the first step, an alcohol feedstock comprising one or more alcohols is dehydrated in the presence of γ-alumina catalyst to form an olefin mixture. In a subsequent step, the olefin mixture is combined with up to two co-monomers with a catalyst system under process conditions to form an oligomer product comprising dimers, trimers, and higher oligomers. In a subsequent step, the oligomer product is hydrogenated to produce a fully saturated branched hydrocarbon. For example, in one embodiment, ethanol is dehydrated to ethylene and included in the olefin mixture.

Other objects and features will be in part apparent and in part pointed out hereinafter.

### BRIEF DESCRIPTION OF THE DRAWING

FIG. 1 illustrates one embodiment of a process for the generation of base oils (e.g., PAOs).

FIG. 2 illustrates one embodiment of a two-stage oligomerization process for the generation of base oils (e.g., PAOs).

FIG. 3 illustrates one embodiment of a process for the generation of PAOs from long-chain alcohols. Exemplary light base oil includes oils with 2 cSt. Exemplary mid-base oil includes oil with 4 cSt, 6 cSt, or 8 cSt. Exemplary heavy base oil includes oil with 7 cSt, 9 cSt, 12 cSt, 17 cSt, or 20 cSt.

FIG. 4 illustrates one embodiment of a process for the generation of PAOs from long-chain alcohol-derived olefins (e.g., linear alpha olefins (LAOs)), and olefin co-monomers. Exemplary light base oil includes oils with 2 cSt. Exemplary mid-base oil includes oils with 4 cSt, 6 cSt, or 8 cSt. Exemplary heavy base oils include oils with 7 cSt, 9, cSt, 12 cSt, 17 cSt, or 20 cSt.

FIG. 5 illustrates one embodiment of a process for the generation of LAOs from ethanol, for example, an ETO (Ethanol to Olefin) process.

FIG. 6 illustrates one embodiment of a process for the generation of LAOs from long-chain alcohols, for example, an ATO (Alcohol to Olefin) process using primary alcohols.

FIG. 7 illustrates one embodiment of an oligomerization process.

FIG. 8 is a schematic of one embodiment of a pilot dehydration reactor train.

FIG. 9 is a schematic of another embodiment of a pilot dehydration reactor train.

FIG. 10A and FIG. 10B show an embodiment of a polymodal oligomer product distribution plot derived from the inventive subject matter disclosed herein. Higher boiling points and increased carbon numbers are indicated along the x-axis. A—3.9 to 4.1 cSt, and average carbon number is approximately C30; B—4.8 to 5.25 cSt, and average carbon number is approximately C30; C—monomer range; D—C8 to C12 dimer range; E—C8 to C12 trimer range; F—C14 to C16 dimer range; G—C14 to C16 trimer range; H—tetramer and higher oligomer range.

FIG. 11 is a schematic of an embodiment of a prior art distillation. In one embodiment of the prior art, the un-reacted alphaolefin and dimers of said alphaolefin are distilled off using a fractional distillation column. In a subsequent step the bottom products is further fractionated into a dimer cut (D1) and trimer cut (D2) and a bottoms product, predominantly trimer and tetramer, which according to one embodiment is no more than 10 cSt, also using a fractional distillation column.

FIG. 12 is a schematic of an embodiment of a C8-C16 distillation related to the inventive subject matter disclosed herein. According to one embodiment, oligomer product is passed to a distillation column to remove and/or recycle the unreacted olefin monomer (D1) and the bottoms (R1) are passed to a 2^{nd}, 3^{rd} and 4^{th} distillation stage which can each be a fractional distillation column or alternatively a short-path evaporator. In a second stage a predominately dimer cut (D2) is taken overhead, typically 2-4 cSt and in the third and 4^{th} stage an early dimer and predominately trimer product is taken overhead (D3 and D4). In one embodiment D3 is up to 4 cSt and D4 is typically 5 cSt or more, and R4 can be between 20 and 20 cSt.

FIG. 13A shows an embodiment of a prior art 28-day biodegradability study using the OECD 301b method for a commercial 4 cSt PAO. The study shows a mean 48.6% degradation in 28 days

FIG. 13B shows an embodiment of a plot characterizing a 4 cSt commercial PAO base oil degradation in 28 days.

FIG. 14A shows an embodiment of a 28-day biodegradability study related to the inventive subject matter disclosed herein using the OECD 301b method. The study shows a mean 74.2% degradability in 28 days.

FIG. 14B shows an embodiment of a plot characterizing 4 cSt hydrocarbon base oil (e.g, using 50% LAO and 50% terpene co-monomers) related to the inventive subject matter disclosed herein.

FIG. 15A shows an embodiment of a 28-day and a 49-day biodegradability study related to the inventive subject matter disclosed herein using the OECD 301b method for a commercial 4 cSt PAO. The study shows a mean 90.3% degradation in 49 days.

FIG. 15B shows an embodiment of a plot characterizing 5 cSt hydrocarbon base oil (e.g., using 50% LAO and 50% terpene co-monomers) related to the inventive subject matter disclosed herein.

Corresponding reference characters indicate corresponding parts throughout the drawings.

### Definitions

“Base oil” as used herein is an oil used to manufacture products including dielectric fluids, hydraulic fluids, compressor fluids, engine oils, lubricating greases, and metal processing fluids.

“Biobased base oil” as used herein is any base oil derived from renewable compositions (e.g., a natural alcohol such as a fatty alcohol).

“Fatty acid” as used herein is a carboxylic acid with a long aliphatic tail (i.e., chain), which is either saturated or unsaturated. Most naturally occurring fatty acids have a chain with an even number of carbon atoms, for example, from 4 to 28.

“Fatty alcohol” as used herein is a high-molecular-weight, straight-chain or branched chain primary alcohol, and may range from as few as 4 carbons to as many as 28 carbons. Fatty alcohols may be derived from natural fats and oils, or fatty acids as described herein.

“Primary alcohol” as used herein means an organic compound having a hydrocarbon chain (e.g., CₙH₂ₙ) terminating with a hydroxyl (—OH) functional group. Non-limiting examples of primary alcohols include n-butanol or isobutanol (C4), 1-pentanol, isoamyl alcohol, or 2-methyl-1-butanol (C5), 1-hexanol (C6), 1-heptanol (C7), 1-octanol or phenethyl alcohol (C8), 1-nonanol (C9), 1-decanol or tryptophol (C10), undecanol (C11), dodecanol (C12), tridecan-1-ol (C13), 1-tetradecanol (C14), 1-pentadecanol (C15), cetyl alcohol (C16).

“Renewable” as used herein means any biologically derived composition, including fatty alcohols, olefins, or oligomers. Such compositions may be made, for nonlimiting example, from biological organisms designed to manufacture specific oils, as discussed in WO 2012/141784, but do not include petroleum distilled or processed oils such as, for non-limiting example, mineral oils. A suitable method to assess materials derived from renewable resources is through “Standard Test Methods for Determining the Biobased Content of Solid, Liquid, and Gaseous Samples Using Radiocarbon Analysis” (ASTM D6866-12 or ASTM D6866-11). Counts from ¹⁴C in a sample can be compared directly or through secondary standards to SRM 4990C. A measurement of 0% ¹⁴C relative to the appropriate standard indicates carbon originating entirely from fossils (e.g., petroleum based). A measurement of 100% ¹⁴C indicates carbon originating entirely from modern sources (See, e.g., WO 2012/141784, incorporated herein by reference).

“Sesquiterpene” as used herein is a class of terpenes that consist of three isoprene units and have the empirical formula C₁₅H₂₄. Sesquiterpenes may be acyclic or contain rings.

“Terpenes” as used herein means biosynthetic units of isoprene (e.g., (C₅H₈)ₙ, where n is the number of linked isoprene units). Representative examples of terpenes (or terpenoids) include, but are not limited to, monoterpenes, partially hydrogenated monoterpenes, sesquiterpenes, and the like.

“Terpene” as used herein is a compound that is capable of being derived from isopentyl pyrophosphate (IPP) or dimethyl allyl pyrophosphate (DMAPP), and the term terpene encompasses hemiterpenes, monoterpenes, sesquiterpenes, diterpenes, sesterterpenes, triterpenes, tetraterpenes, and polyterpenes. A hydrocarbon terpene contains only hydrogen and carbon atoms and no heteroatoms such as oxygen, and in some embodiments has the general formula (C₅H₈)ₙ, where n is 1 or greater. A “conjugated terpene” or “conjugated hydrocarbon terpene” as used herein refers to a terpene comprising at least one conjugated diene moiety. It should be noted that the conjugated diene moiety of a conjugated terpene may have any stereochemistry (e.g., cis or trans, or E or Z)) and may be part of a longer conjugated segment of a terpene, for example, the conjugated diene moiety may be part of a conjugated triene moiety. It should be understood that hydrocarbon terpenes as used herein also encompasses monoterpenoids, sesquiterpenoids, diterpenoids, triterpenoids, tetraterpenoids and polyterpenoids that exhibit the same carbon skeleton as the corresponding terpene, but have either fewer or additional hydrogen atoms than the corresponding terpene, for example, terpenoids having 2 fewer, 4 fewer, or 6 fewer hydrogen atoms than the corresponding terpene, or terpenoids having 2 additional, 4 additional, or 6 additional hydrogen atoms than the corresponding terpene. The terms “terpene” and “isoprenoids” are used interchangeably herein, and are a large and varied class of organic molecules that can be produced by a wide variety of plants and some insects. Some terpenes or isoprenoid compounds can also be made from organic compounds such as sugars by microorganisms, including bioengineered microorganisms. Because terpenes or isoprenoid compounds can be obtained from various renewable sources, they are useful monomers for making eco-friendly and renewable base oils.

“Olefin co-monomer” refers to any olefin containing at least one carbon-carbon double bond. “Olefin co-monomer(s)” means one or more olefin co-monomers, where it is understood that two olefin co-monomers refers to two olefin co-monomers that are different from each other, etc.

“Alpha-olefin” as used herein refers to any olefin having at least one terminal, unconjugated carbon-carbon double bond. “Alpha-olefin” encompasses linear alpha-olefins (LAOs) and branched alpha-olefins. Alpha-olefins may contain one or more carbon-carbon double bonds in addition to the terminal olefinic bond, for example, alpha, omega-dienes.

“Linear internal olefins (LIOs)” as used herein refers to linear olefins containing one or more carbon-carbon double bonds, none of which are located at a terminal position. “Branched internal olefins” as used herein refers to branched olefins containing one or more carbon-carbon double bonds, none of which are located at a terminal position.

“Oligomer” as used herein refers to a molecule having 2-100 monomeric units, and encompasses dimers, trimers, tetramers, pentamers, and hexamers. An oligomer may comprise one type of monomer unit or more than one type of monomer unit, for example, two types of monomer units, or three types of monomer units. “Oligomerization” as used herein refers to the formation of a molecule having 2-100 monomeric units from one or more monomers, and encompasses dimerization, trimerization, etc. of one type of monomer, and also encompasses the formation of adducts between more than one type of monomer.

“Polymer” as used herein refers to a polymeric compound prepared by polymerizing monomers, whether of the same or a different type, and having more than 100 monomeric units. The generic term “polymer” embraces the terms “homopolymer,” “copolymer,” “terpolymer” as well as “interpolymer.” The generic term “interpolymer” encompasses the term “copolymer” (which generally refers to a polymer prepared from two different monomers) as well as the term “terpolymer” (which generally refers to a polymer prepared from three different types of monomers), and polymers made by polymerizing four or more types of polymers.

“Dimer” or “dimeric species” as used herein refers to any type of adducts formed between two molecules, and encompasses 1:1 adducts of the same types of molecules or 1:1 adducts of different types of molecules, unless specifically stated otherwise. “Trimer” or “trimeric species” as used herein refers to any type of adducts formed between three molecules, and encompasses 1:1:1 of the same types of molecules or three different types of molecules, and 1:2 or 2:1 adducts of two different types of molecules. “Tetramer” or “tetrameric species” as used herein refers to any type of adducts formed between four molecules. “Pentamer” or “pentameric species” as used herein refers to any type of adducts formed between five molecules. “Hexamer” or “hexameric species” as used herein refers to any type of adducts formed between six molecules.

“Viscosity index” as used herein refers to viscosity index as measured according to “Standard Practice for Calculating Viscosity Index From Kinematic Viscosity at 40 and 100° C.” (ASTM D2270) published by ASTM International, which is incorporated herein by reference in its entirety. Kinematic viscosities at 40° C. and at 100° C. are measured according to “Standard Test Method for Kinematic Viscosity of Transparent and Opaque Liquids (and Calculation of Dynamic Viscosity)” (ASTM D445) published by ASTM International, which is incorporated herein by reference in its entirety.

“Pour point” is measured according to “Standard Test Method for Pour Point of Petroleum Products” (ASTM D97) published by ASTM International, which is incorporated herein by reference in its entirety.

“Cold cranking simulator viscosity” as used herein refers to cold cranking simulator viscosity as measured according to “Standard Test Method for Apparent Viscosity of Engine Oils Between −5 and −35° C. Using the Cold-Cranking Simulator” (ASTM D5293) published by ASTM International, which is incorporated herein by reference in its entirety.

“Boiling point” refers to the natural boiling point of a substance at atmospheric pressure, unless indicated otherwise. Simulated Distillation may be carried out according to “Standard Test Method for Boiling Range Distribution of Petroleum Distillates in Boiling Range from 174° C. to 700° C. by Gas Chromatography” (ASTM D 6352—02), “Test Method for Boiling Range Distribution of Petroleum Fractions by Gas Chromatography” (ASTM D2887), or “Standard Test Method for Estimation of Engine Oil Volatility by Capillary Gas Chromatography” (ASTM D 6417), each published by ASTM International, and each of which is incorporated herein by reference in its entirety.

Evaporative weight loss may be carried out according to “Standard Test Method for Evaporation Loss of Lubricating Oils by the Noack Method” (ASTM D5800), or “Standard Test Method for Evaporation Loss of Lubricating Oils by Thermogravimetric Analyzer (TGA) Noack Method” (ASTM D6375, TGA-Noack method), each published by ASTM International, and each of which is incorporated herein by reference in its entirety.

The degree of unsaturation of a product, such as a hydrogenated oligomer product, can be quantified according to the Bromine Index of the product, as determined in accordance with ASTM D2710-09, which is incorporated by reference herein in its entirety.

In the following description, all numbers disclosed herein are approximate values, regardless of whether the word “about” or “approximate” is used in connection therewith. Numbers may vary by 1%, 2%, 5% or sometimes 10 to 20%. Whenever a numerical range with a lower limit R_{L} and an upper limit R_{U} is disclosed, any number falling within the range is specifically disclosed. In particular, the following numbers Rₖ within the range are specifically disclosed: Rₖ=R_{L}+k*(R_{U}−R_{L}), wherein k is a variable ranging from 1% to 100% with a 1% increment (i.e., k is 1 percent, 2 percent, 3 percent, 4 percent, 5 percent, . . . 50 percent, 51 percent, 52 percent, . . . 95 percent, 96 percent, 97 percent, 98 percent, 99 percent, or 100 percent). Further, any numerical range defined by any two numbers Rₖ as defined above is also specifically disclosed herein.

As used herein and unless otherwise indicated, a reaction that is “substantially complete” means that the reaction contains more than about 80% desired product by percent yield, more than about 90% desired product by percent yield, more than about 95% desired product by percent yield, or more than about 97% desired product by percent yield. As used herein, a reactant that is “substantially consumed” means that more than about 85%, more than about 90%, more than about 95%, more than about 97% of the reactant has been consumed, by weight %, or by mol %. As used herein, % refers to % measured as wt. % or as area % by GC-MS or GC-FID, unless specifically indicated otherwise.

As used herein and unless otherwise indicated, a composition that is made up “predominantly” of a particular component includes at least about 60% of that component. A composition that “consists essentially of” a component refers to a composition comprising 80% or more of that component, unless indicated otherwise.

Unless otherwise stated herein, all concentration percentages shall be understood to be on a weight percent basis.

### DETAILED DESCRIPTION

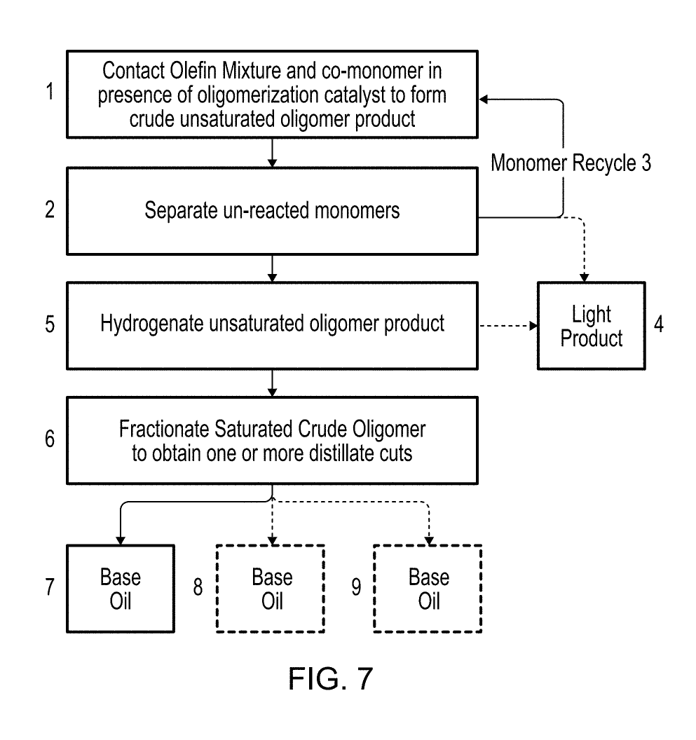

Referring now to FIG. 7, one aspect of the present disclosure is an oligomerization process. Per this embodiment, in Step 1 an oligomerization reaction mixture comprising an oligomerization catalyst, a population of olefins and, optionally, co-monomer(s), is provided in an oligomerization reactor, and an oligomerization reaction product containing a crude oligomer product is formed. In Step 2, unreacted monomer is separated from the oligomerization reaction product and optionally recycled (Step 3) to the oligomerization reactor, and the crude unsaturated oligomer product is delivered to a hydrogenation reactor (Step 5) to form a hydrogenated reaction product. From there, the hydrogenated reaction product may be fractionated by distillation (Steps 4 and 6) to obtain one or more distillate cuts and provide one or more base oil products (Steps 7, 8, and 9). As described in greater detail elsewhere herein, the population of olefins or one or more of the optional co-monomers may comprise renewable carbon derived, for example, from one or more alcohols (e.g., ethanol or a fatty alcohol) or from one or more fatty acids. Alternatively, or additionally, the population of olefins or the co-monomers may comprise one or more alkenes such as 1-octene, 1-decene or 1-dodecene derived from petroleum or other non-renewable sources.

In certain embodiments, the process of the present disclosure may be used to form biobased base oils. For example, in one such embodiment, at least about 10% of the carbon atoms in the base oil originate from renewable carbon sources. By way of further example, in one such embodiment, at least about 20% of the carbon atoms in the base oil originate from renewable carbon sources. By way of further example, in one such embodiment, at least about 30% of the carbon atoms in the base oil originate from renewable carbon sources. By way of further example, in one such embodiment, at least about 40% of the carbon atoms in the base oil originate from renewable carbon sources. By way of further example, in one such embodiment, at least about 50% of the carbon atoms in the base oil originate from renewable carbon sources. By way of further example, in one such embodiment, at least about 60% of the carbon atoms in the base oil originate from renewable carbon sources. By way of further example, in one such embodiment, at least about 70% of the carbon atoms in the base oil originate from renewable carbon sources. By way of further example, in one such embodiment, at least about 80% of the carbon atoms in the base oil originate from renewable carbon sources. By way of further example, in one such embodiment, at least about 90% of the carbon atoms in the base oil originate from renewable carbon sources. In some variations, the carbon atoms of the base oil comprise at least about 95%, at least about 97%, at least about 99%, or about 100% of originate from renewable carbon sources. By way of further example, in one such embodiment, at least about 90% of the carbon atoms in the base oil originate from renewable carbon sources. In some variations, the carbon atoms of the base oil comprise less than 100% of originate from renewable carbon sources. In some variations, the carbon atoms of the base oil comprise less than 95%, or even less than 90%. In some variations, about 10% to about 90% of the carbon atoms of the base oil are from renewable carbon sources. The origin of carbon atoms in the reaction product adducts may be determined by any suitable method, including but not limited to reaction mechanism combined with analytical results that demonstrate the structure and/or molecular weight of adducts, or by carbon dating (e.g., according to “Standard Test Methods for Determining the Biobased Content of Solid, Liquid, and Gaseous Samples Using Radiocarbon Analysis” (ASTM D6866-12), which is incorporated herein by reference in its entirety). For example, using ASTM D6866-12 or another suitable technique, a ratio of carbon 14 to carbon 12 isotopes in the biobased base oil can be measured by liquid scintillation counting and/or isotope ratio mass spectroscopy to determine the amount of modern carbon content in the sample. A measurement of no modern carbon content indicates all carbon is derived from fossil fuels. A sample derived from renewable carbon sources will indicate a concomitant amount of modern carbon content, up to 100%

In some embodiments of this disclosure, one or more repeating units of a biobased hydrocarbon base oil is a specific species of partially hydrogenated, conjugated hydrocarbon terpenes. Such specific species of partially hydrogenated, conjugated terpenes may or may not be produced by a hydrogenation process. In certain variations, a partially hydrogenated, conjugated hydrocarbon terpene species is prepared by a method that includes one or more steps in addition to or other than catalytic hydrogenation. Non-limiting examples of specific species of partially hydrogenated, conjugated hydrocarbon terpenes include sesquiterpenes, dihydromyrcene, tetrahydromyrcene, dihydroocimene, and tetrahydroocimene.

In certain embodiments, the oligomer product may be isomerized during the hydrogenation step. Isomerizations may include the generation of E- or Z-mixtures of olefins in a biobased hydrocarbon base oil. Isomerizations may also include the generation of E- and Z-olefins within a biobased hydrocarbon base oil. For example, in one embodiment, during the hydrogenation step, the oligomer product may be isomerized into an all Z-olefin mixture. By way of further example, in one embodiment, during the hydrogenation step, the oligomer product may be isomerized into an all E-olefin mixture.

In some embodiments, the present disclosure includes a process for the generation of polyalphaolefins (PAOs) from alcohol-derived feedstocks. The process may include a feedstock composition, a first olefinic mixture, an optional second olefinic mixture, an oligomerization, a distillation, a hydrogenation, a separation, and a final base oil composition.

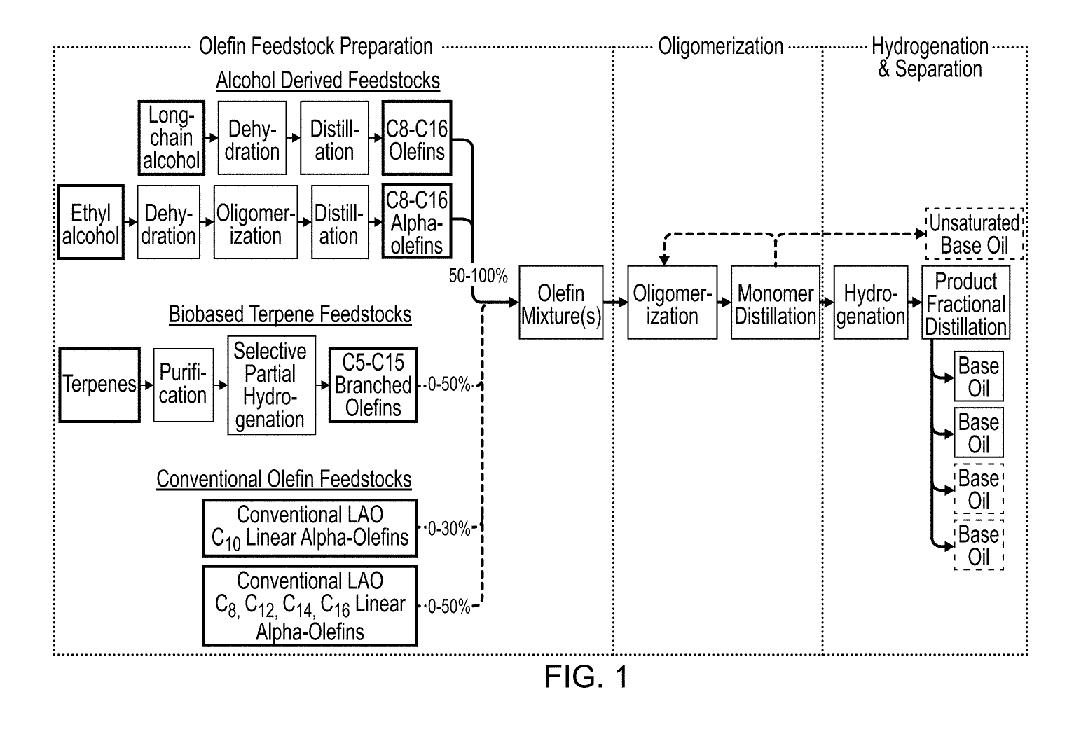

Referring now to FIG. 1, a process for the generation of PAOs includes an olefin feedstock composition (sometimes referred to as the “olefin mixture(s)” as illustrated in FIG. 1). In general, the olefin feedstock composition includes a population of olefins derived from any of three sources: (1) alcohol-derived olefin populations; (2) biobased terpene populations; and (3) conventional olefin populations derived from non-renewable sources. Exemplary alcohols for the alcohol-derived olefins include primary alcohols, secondary alcohols, tertiary alcohols, or combinations thereof. For example, in one embodiment, the olefin feedstock comprises a population of olefins derived from C2-C16 primary alcohols selected from the group consisting of ethanol, n-butanol, 1-pentanol, 1-hexanol, 1-heptanol, 1-octanol, 1-nonanol, 1-decanol, 1-undecanol, 1-dodecanol, 1-tridecanol, 1-tetradecanol, 1-pentadecanol, 1-hexadecanol, isoamyl alcohol, 2-methyl-1-butanol, phenethyl alcohol, tryptophol, and combinations thereof (e.g., 50-100 wt % of the olefin mixture (i.e., the olefin reaction mixture) for the oligomerization reaction) By way of further example, in one embodiment, the olefin feedstock comprises a population of olefins derived from C3-C7 secondary alcohols selected from the group consisting of isopropanol, 2-butanol, 2-pentanol, 2-hexanol, 2-heptanol, cyclohexanol, and combinations thereof. By way of further example, in one embodiment, the olefin feedstock comprises a population of olefins derived from C4-C9 tertiary alcohols selected from the group consisting of tert-butanol, tert-amyl alcohol, 2-methyl-2-pentanol, 2-methylhexan-2-ol, 2-methylheptan-2-ol, 3-methyl-3-pentanol, 3-methyloctan-3-ol. Exemplary olefins within the feedstock composition may also include terpenes and conventional olefins. For example, in one embodiment, the feedstock composition further includes C5-C15 biobased terpenes. By way of further example, in one embodiment, C5-C15 biobased terpenes may be selected from the group consisting of isoprene, monoterpenes, partially hydrogenated monoterpenes, sesquiterpenes, partially hydrogenated sesquiterpenes, and combinations thereof. By way of further example, in one embodiment, the feedstock composition further includes C8-C16 conventional olefins. By way of further example, C8-C16 conventional olefins may be selected from the group consisting of 1-octene, 1-decene, 1-dodecene, 1-tetradecene, 1-hexadecene, and combinations thereof.

As illustrated in FIG. 1, in certain embodiments the olefin feedstock (i.e., the olefin mixture as illustrated in FIG. 1) comprises as a percentage of the olefin mixture, 50 to 100% olefins derived from a short chain alcohol such as ethanol or a long chain (fatty alcohol) mixture. The long chain alcohols may be, for example, any of the alcohols previously described herein. In certain embodiments, the long chain alcohols may be selected from 1-octanol, 1-dodecanol, and combinations thereof. In certain embodiments, the olefin feedstock may optionally comprise 0-50% biobased terpenes (as a weight percentage of the olefins comprised by the olefin mixture) and/or 0-30% conventional olefin feedstocks (as a weight percentage of the olefins comprised by the olefin mixture). In general, however, certain conventional olefin feedstocks such as 1-decene are less preferred in certain embodiments. In such embodiments, therefore, the olefin feedstock comprises less than 25% (as a weight percentage of the olefins comprised by the olefin mixture). By way of further example, in one such embodiment, the olefin feedstock comprises less than 20% (as a weight percentage of the olefins comprised by the olefin mixture). By way of further example, in one such embodiment, the olefin feedstock comprises less than 15% (as a weight percentage of the olefins comprised by the olefin mixture). By way of further example, in one such embodiment, the olefin feedstock comprises less than 10% (as a weight percentage of the olefins comprised by the olefin mixture). By way of further example, in one such embodiment, the olefin feedstock comprises less than 5% (as a weight percentage of the olefins comprised by the olefin mixture). By way of further example, in one such embodiment, the olefin feedstock comprises less than 1% (as a weight percentage of the olefins comprised by the olefin mixture). In each of the foregoing embodiments, the olefin feedstock may have an average carbon number in the range of 9.5 to 13, such as in the range of 9.5 to 10.5, and even in the range of 9.9 to 10.5, such as in the range of 10.6 to 13.

In one exemplary embodiment, the olefin feedstock comprises 0-25% 1-decene, 25-50% 1-octene, and 15-50% 1-dodecene. In one such embodiment, the 1-octene comprises renewable carbon. In another such embodiment, the 1-dodecene comprises renewable carbon. In yet another such embodiment, the 1-octene and the 1-dodecene each comprise renewable carbon. As previously noted, certain conventional olefin feedstocks such as 1-decene are less preferred in certain embodiments. In each of the foregoing embodiments, therefore, the olefin feedstock preferably comprises less than 25% 1-decene (as a weight percentage of the olefins comprised by the olefin mixture). By way of further example, in each of the foregoing embodiments, the olefin feedstock may comprise less than 20% 1-decene (as a weight percentage of the olefins comprised by the olefin mixture). By way of further example, in each of the foregoing embodiments, the olefin feedstock may comprise less than 15% 1-decene (as a weight percentage of the olefins comprised by the olefin mixture). By way of further example, in each of the foregoing embodiments, the olefin feedstock may comprise less than 10% 1-decene (as a weight percentage of the olefins comprised by the olefin mixture). By way of further example, in each of the foregoing embodiments, the olefin feedstock may comprise less than 5% 1-decene (as a weight percentage of the olefins comprised by the olefin mixture). By way of further example, in each of the foregoing embodiments, the olefin feedstock may comprise less than 1% 1-decene (as a weight percentage of the olefins comprised by the olefin mixture). By way of further example, in each of the foregoing embodiments, the olefin feedstock may have an absence of 1-decene. In each of the foregoing embodiments, the olefin feedstock may have an average carbon number in the range of 9.5 to 13, such as in the range of from 9.5 to 10.5, and even in the range of from 9.9 to 10.5, such as in the range of from 10.6 to 13.

FIG. 1 further shows a process for the preparation of branched saturated hydrocarbons, the process comprising a first step of forming at least one olefin feedstock mixture. The olefin feedstock mixture is comprised of
(a) From 10-90% of composition A, alcohol derived olefins. Olefin composition A consists of one or more ethyl alcohol or long-chain alcohol derived olefins. The ethyl alcohol derived olefins are made by dehydration of ethyl alcohol to ethylene, followed by a catalytic oligomerization to form a linear alpha-olefin product as disclosed in the prior art, for example, the Ineos (Ethyl) process “Ethylene chain growth process,” U.S. Pat. No. 5,049,687 A, and references cited therein. The long-chain alcohol derived olefins are made by the dehydration of alcohols, preferably primary alcohols, over a gamma alumina catalyst 0.1-45 PSIA (psi at atmospheric pressure) at 250-350° C. to form C8-C16 linear alpha-olefins;
(b) From 0-50% of composition B, terpene derived olefins. Terpene derived olefins can be any biologically or biosynthetic terpenoids which have been partially hydrogenated to produce predominately mono-olefins, preferably a partially hydrogenated sesquiterpene (e.g., C15);
(c) Less than 25% conventional 1-decene linear alpha-olefin derived from ethylene; and/or
(d) Not more than 90% conventional linear alpha-olefin selected from C8, C12, C14, or C16.

A second step includes where the olefin mixture is charged to the first stage oligomerization reactor and oligomerized. The reaction is carried out in the presence of a suitable oligomerization catalyst. In one embodiment, the olefin mixture may be treated to remove impurities prior to the oligomerization step.

In a subsequent step optionally a two-stage reaction may be practiced where a second olefin mixture having a different composition than the first olefin mixture is charged to a second stage oligomerization reactor along with the product from the first stage reactor whereupon a second oligomerization catalyst is charged and a second oligomer product is formed.

In a subsequent step the reaction product is discharged and the un-reacted monomer or lights are distilled, in part or in full, and recycled with an optional off-take of the unsaturated lights as a separate product stream.

In a subsequent step, the stripped oligomer product is hydrogenated in either a continuous flow reactor or a batch stirred tank reactor using a nickel (Ni) catalyst, as is known in the art.

In a final step, the hydrogenated oligomer is fractionally distilled using one or more fractional distillation columns and one or more short-path evaporators. In general, long-chain alcohols may be dehydrated, followed by a distillation, that yields a mixture of C8-C16 olefins. Alternatively, in general, ethyl alcohol may be dehydrated, oligomerized, and distilled to provide a mixture of C8-C16 alpha-olefins.

In general, terpenes may be purified and subjected to selective partial hydrogenation to provide a mixture of C8-C16 alpha-olefins.

Oligomerizations typically use suitable catalytic conditions under suitable temperatures to generate PAOs. For example, suitable catalysts used in oligomerizations include Friedel-Crafts catalysts and metallocene catalysts. Exemplary Friedel-Crafts catalysts include Group 13 elements. For example, in one embodiment, the catalyst may be selected from the group consisting of boron trifluoride, aluminum trichloride, gamma-alumina, and combinations thereof. Exemplary metallocene catalysts include titanocenes, zirconocenes, hafnocenes, and the like, and combinations thereof. In some embodiments, suitable co-catalysts may also be used for oligomerizations. Suitable co-catalysts include alcohols, alkyl acetates, methylaluminoxane, and the like. For example, suitable alcohol co-catalysts include C1-C10 alcohols. By way of further example, suitable alcohol co-catalysts include C1-C6 alcohols selected from the group consisting of methanol, ethanol, n-propanol, n-butanol, n-pentanol, n-hexanol, and combinations thereof. By way of further example, suitable alkyl acetate co-catalysts include C1-C10 alkyl acetates. By way of further example, suitable C1-C6 alkyl acetates selected from the group consisting of methyl acetate, ethyl acetate, n-propyl acetate, n-butyl acetate, and combinations thereof. In any of the above embodiments, suitable catalysts and/or cocatalysts may be used in amounts known to those of skill in the art to provide oligomerization products, such as PAOs. Suitable temperatures for oligomerization are also known to those of skill in the art. For example, in one embodiment, the oligomerization temperature can vary from about −20° C. to about 90° C. By way of further example, in one embodiment, the oligomerization temperature can vary from about 15° C. to about 70° C.

In some embodiments, distillations following oligomerizations are used to remove unreacted olefin monomers. In other embodiments, distillations are used to remove unreacted monomers and dimers. In yet other embodiments, distillations are used to further remove dimers.

In some embodiments, hydrogenations of purified oligomers are used to saturate remaining trimers and higher oligomers. Conventional hydrogenation conditions are known to those of skill in the art. For example, in certain embodiments, typical hydrogenations include hydrogenation catalysts. By way of further example, in some embodiments, hydrogenation catalysts may be selected from the group consisting of palladium, platinum, nickel, and the like, and combinations thereof.

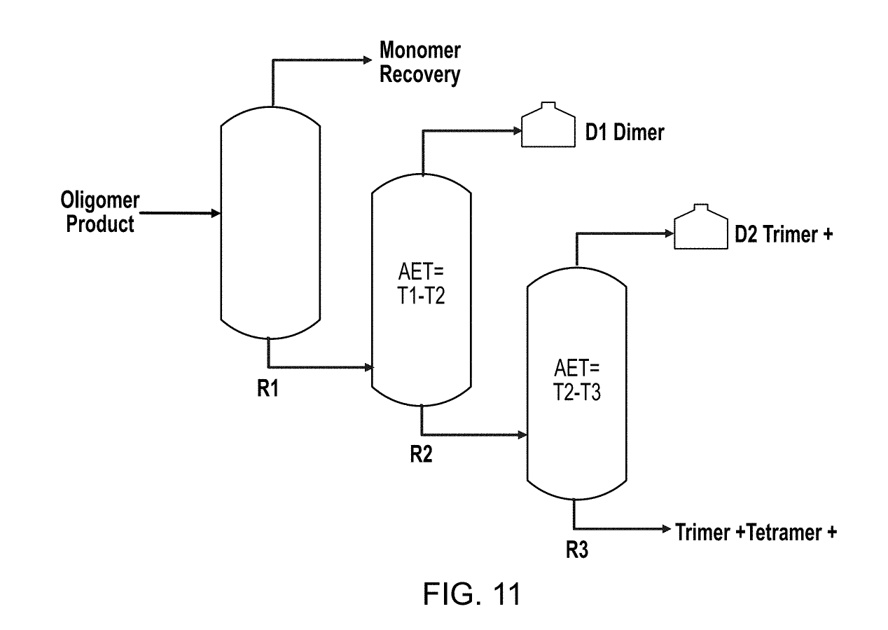

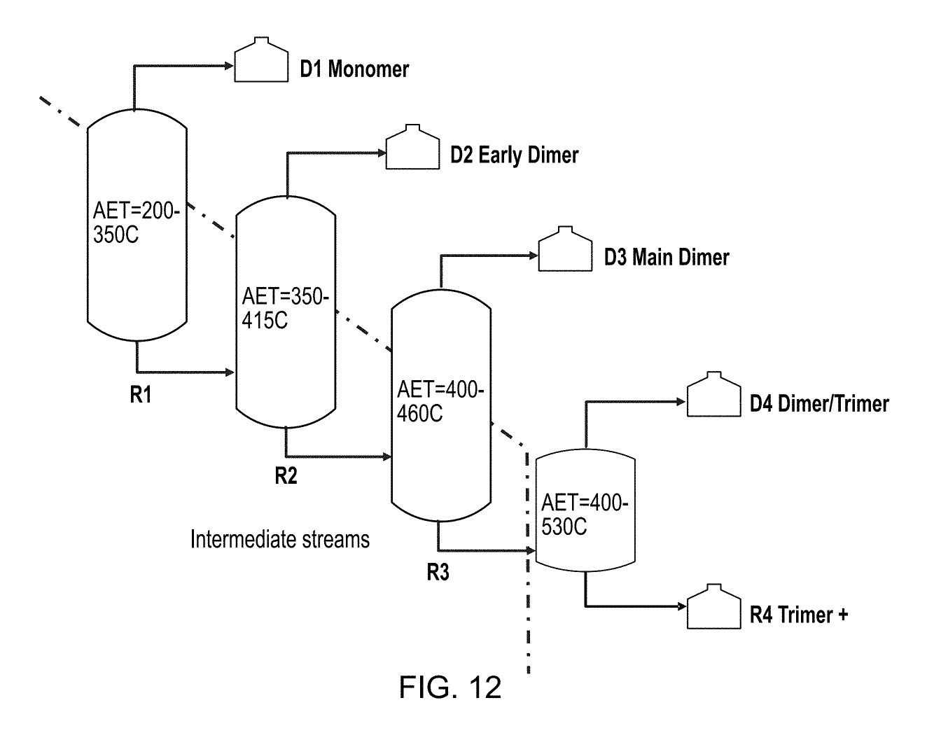

In some embodiments, a separation includes a plurality of distillations to provide the final base oil. For example, in some embodiments, distillations may include a plurality of fractional distillations as shown in FIG. 12; for comparison, distillation according to a prior art method is shown in FIG. 11.

In some embodiments, the final base oil composition has favorable PAO properties for use as lubricants, and the like. Favorable PAO properties for the base oils generated in the process described herein are dependent on the feedstock composition described herein and may include low Noack volatilities, low kinematic viscosities, and low pour points. Exemplary low Noack volatilities, in one embodiment, include a range of about 10% to about 15% weight loss. By way of further example, in one embodiment, low Noack volatilities include a range of about 11% to about 14% weight loss. Noack volatility is typically determined via the ASTM D5800 method, as known to those of skill in the art, and incorporated herein by reference in its entirety. Exemplary low kinematic viscosities, in one embodiment, include about 6 cSt at 100° C. By way of further example, in one embodiment, low kinematic viscosities include about 4 cSt at 100° C. By way of further example, in one embodiment, low kinematic viscosity may range from at least about 45% of 4 cSt PAO to not more than about 55% of 6 cSt PAO. By way of further example, in one embodiment, low kinetic viscosity may include equal amounts of 4 cSt and 6 cSt PAOs. By way of further example, in one embodiment, low kinetic viscosity may include higher amounts of 4 cSt compared to amounts of 6 cSt. Exemplary low pour points, in one embodiment, may include about −45° C. to about −80° C. By way of further example, in one embodiment, low pour points may include about −60° C. to about −70° C. Pour points are typically determined via the ASTM D5950 method, as known to those of skill in the art, and incorporated herein by reference in its entirety.

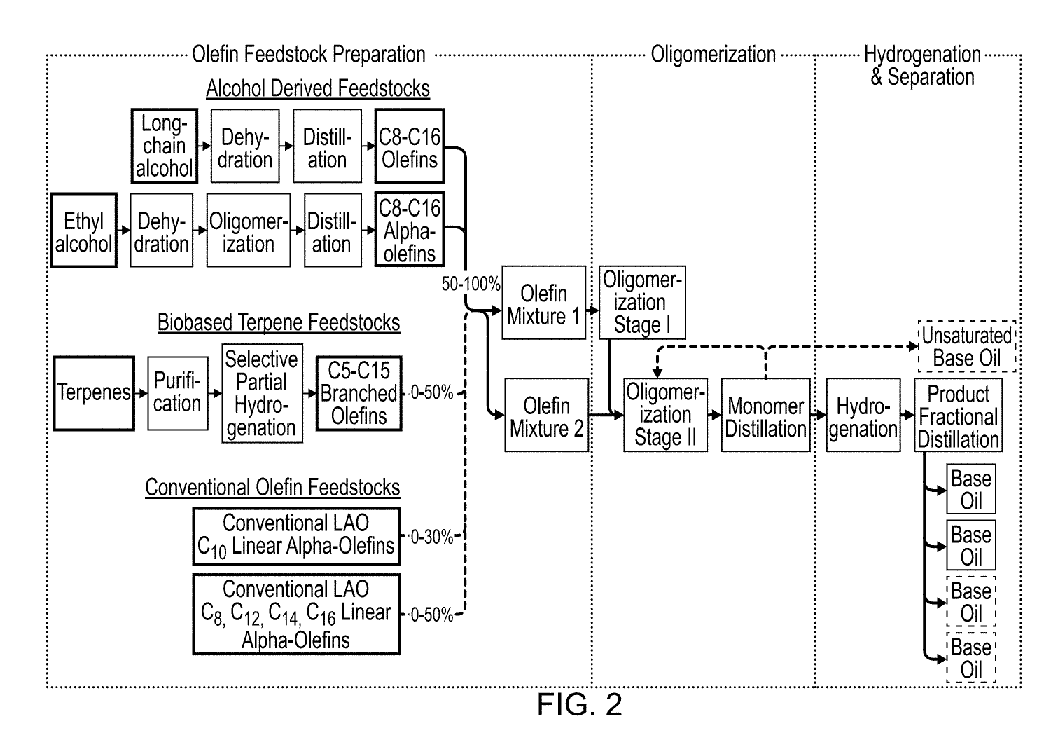

In certain embodiments, a plurality of olefinic mixtures may be generated from alcohol-derived olefins described herein, biobased olefins described herein, conventional olefins described herein, and combinations thereof. For example, a first olefin mixture and a second olefin mixture (see FIG. 2) may be provided for oligomerization.

In some embodiments, the process for the generation of polyalphaolefins (PAOs) from alcohol-derived feedstocks may be performed in a single batch mode or a continuous batch mode.

Referring now to FIG. 2, in some embodiments, the first olefinic mixture may be oligomerized to provide an Oligomerization Stage I mixture to be further oligomerized with the second olefin mixture. For example, the first olefin mixture may be oligomerized to provide an Oligomerization Stage I mixture that is further oligomerized with the second olefin mixture to provide an Oligomerization Stage II mixture. Further processing to base oils is similar to processing as described in FIG. 1.

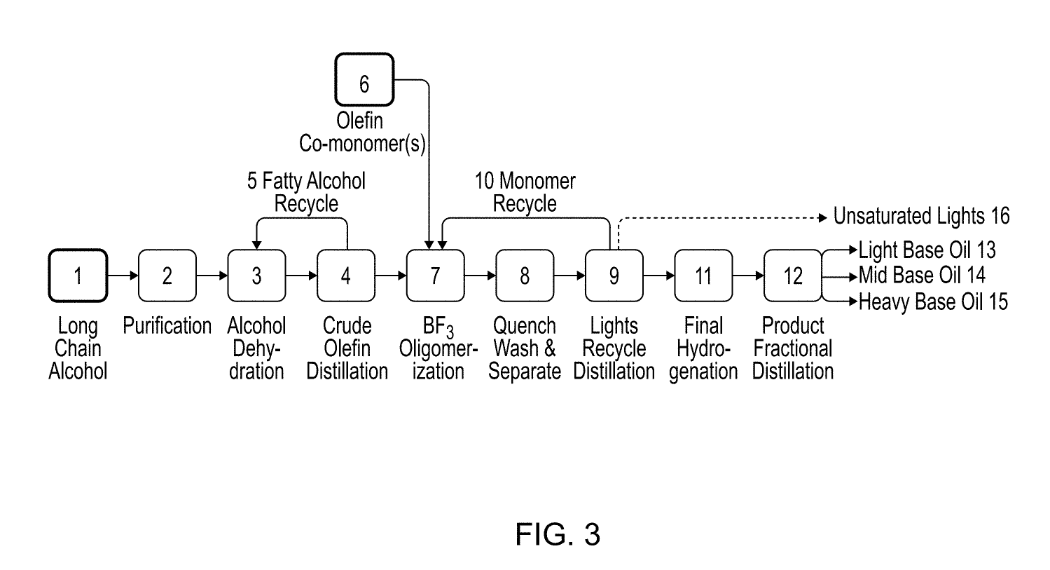

Referring now to FIG. 3, in one embodiment, the present disclosure is further directed to a process for the generation of polyalphaolefins (PAOs) from long-chain alcohols 1. Long-chain alcohols 1 may include primary alcohols, secondary alcohols, and tertiary alcohols. For example, in one embodiment, long-chain alcohols 1 include primary alcohols, secondary alcohols, tertiary alcohols, and combinations thereof. For example, in one embodiment, the long-chain alcohols 1 include C2-C16 primary alcohols selected from the group consisting of ethanol, n-butanol, 1-pentanol, 1-hexanol, 1-heptanol, 1-octanol, 1-nonanol, 1-decanol, 1-undecanol, 1-dodecanol, 1-tridecanol, 1-tetradecanol, 1-pentadecanol, 1-hexadecanol, isoamyl alcohol, 2-methyl-1-butanol, phenethyl alcohol, tryptophol, and combinations thereof. By way of further example, in one embodiment, the long-chain alcohols 1 include C3-C7 secondary alcohols selected from the group consisting of isopropanol, 2-butanol, 2-pentanol, 2-hexanol, 2-heptanol, cyclohexanol, and combinations thereof. By way of further example, in one embodiment, the long-chain alcohols 1 include C4-C9 tertiary alcohols selected from the group consisting of tert-butanol, tert-amyl alcohol, 2-methyl-2-pentanol, 2-methylhexan-2-ol, 2-methylheptan-2-ol, 3-methyl-3-pentanol, 3-methyloctan-3-ol, and combinations thereof. Long chain alcohols 1 are then purified 2 via distillation as described herein, dehydrated 3 as described herein, providing a crude olefin 4 that is further distilled. Distillate from the crude olefin 4 consists of an alcoholic mixture (e.g., fatty alcohols 5) that may be recycled back for dehydration 3. Crude olefin 4 may further undergo BF₃-mediated oligomerization 7, followed by quenching, washing, and separating 8, providing a Lights Recycle mixture 9. Optionally, olefin co-monomers 6 may be added to crude olefin 7 for BF₃-mediated oligomerization en route to Lights Recycle 9. Distillation of Lights Recycle 9 provides a mixture of unreacted monomer 10 for recycling back into BF₃-mediated oligomerization 7, and Unsaturated Lights 16 as by-products. Lights Recycle 9 is finally hydrogenated 11 to provide Product 12 that is further fractionally distilled providing Light Base Oils 13, Mid Base Oils 14, and Heavy Base Oil 15. Exemplary Light Base Oils 13 may include 2 cSt base oil. Exemplary Mid Base Oil may include a range of about 4 cSt to about 8 cSt. By way of further example, in one embodiment, Mid Base oil may include a range of about 4 cSt to about 6 cSt. By way of further example, in one embodiment, Mid Base Oil may include 4 cSt, 6 cSt, or 8 cSt, respectively. Exemplary Heavy Base Oil may include a range of about 7 cSt to about 20 cSt. By way of further example, in one embodiment, Heavy Base Oil may include a range of about 7 cSt to about 17 cSt. By way of further example, in one embodiment, Heavy Base Oil may include a range of about 7 cSt to about 12 cSt. By way of further example, in one embodiment, Heavy Base Oil may include a range of about 7 cSt to about 12 cSt. By way of further example, in one embodiment, Heavy Base Oil may include a range of about 7 cSt to about 9 cSt. By way of further example, in one embodiment, Heavy Base Oil may include 7 cSt, 9 cSt, 12 cSt, 17 cSt, or 20 cSt, respectively.

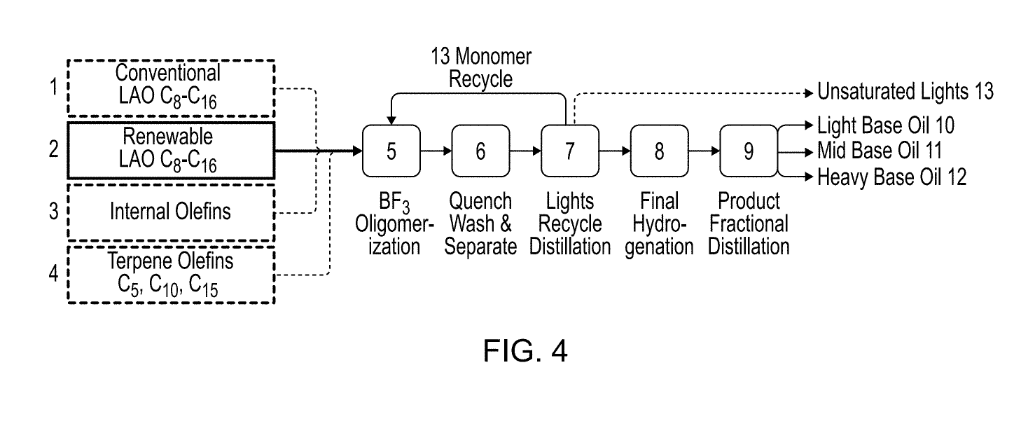

In general, the present disclosure further includes a process for the generation of polyalphaolefins (PAOs) from long-chain alcohol-derived olefins (e.g., linear alpha olefins (LAOs)), and olefin co-monomers. Referring now to FIG. 4, a mixture of olefins may be used as feedstock. Exemplary olefins include conventional LAOs 1, renewable LAOs 2, internal olefins 3, terpenoids 4, and combinations thereof. Exemplary conventional LAOs 1 include C8-C16 conventional LAOs. In one such exemplary embodiment, C8-C16 conventional LAOs may be selected from the group consisting of 1-octene, 1-decene, 1-dodecene, 1-tetradecene, 1-hexadecene, and combinations thereof. Exemplary renewable LAOs 2 include C8-C16 renewable LAOs. In one such exemplary embodiment, C8-C16 renewable LAOs may be selected from the group consisting of 1-octene, 1-decene, 1-dodecene, 1-tetradecene, 1-hexadecene, and combinations thereof. Exemplary internal olefins 3 may be selected from the group consisting of 2-octene, 2-decene, 2-dodecene, 2-tetradecene, 2-hexadecene, including all other olefinic regioisomers, without limitation, and combinations thereof. Exemplary terpenoids 4 may include C5, C10, and/or C15 terpenoids, and combinations thereof. In one such exemplary embodiment, C5, C10, and/or C15 terpenoids may be selected from the group consisting of isoprene, myrcene, farnecene, partially hydrogenated versions thereof, and the like, and combinations thereof. In another exemplary embodiment, C15 terpenoids may include at least one sesquiterpene. For example, in one embodiment, the olefin mixture includes at least one sesquiterpene, but less than 50 wt % sesquiterpene, based upon the weight of the olefins in the olefin mixture. By way of further example, in one embodiment, the olefin mixture includes 5 to 50 wt % sesquiterpene, based upon the weight of the olefins in the olefin mixture. By way of further example, in one embodiment, the olefin mixture includes 10 to 50 wt % sesquiterpene, based upon the weight of the olefins in the olefin mixture. By way of further example, in one embodiment, the olefin mixture includes 15 to 50 wt % sesquiterpene, based upon the weight of the olefins in the olefin mixture. By way of further example, in one embodiment, the olefin mixture includes 25 to 50 wt % sesquiterpene, based upon the weight of the olefins in the olefin mixture. By way of further example, in one embodiment, the olefin mixture includes 10 to 40 wt % sesquiterpene, based upon the weight of the olefins in the olefin mixture. By way of further example, in one embodiment, the olefin mixture includes 25 to 40 wt % sesquiterpene, based upon the weight of the olefins in the olefin mixture. By way of further example, in one embodiment, the olefin mixture includes 10 to 25 wt % sesquiterpene, based upon the weight of the olefins in the olefin mixture.

Olefins 1-4 described above may then be subjected to BF₃-mediated oligomerization 5, followed by quenching, washing, and separating 6, thereby providing Lights Recycle 7. Lights Recycle 7 may then be purified via distillation before final hydrogenation 8. Distillate from Lights Recycle 7 provides unreacted monomer 13 that may be recycled back to BF₃-mediated oligomerization 5, and unsaturated Lights 13 as a by-product. Final hydrogenation 8 then provides Product 9 wherein fractional distillation provides Light Base Oil 10, Mid Base Oil 11, and Heavy Base Oil 12. Exemplary Light Base Oils 13 may include 2 cSt base oil. Exemplary Mid Base Oil may include a range of about 4 cSt to about 8 cSt. By way of further example, in one embodiment, Mid Base oil may include a range of about 4 cSt to about 6 cSt. By way of further example, in one embodiment, Mid Base Oil may include 4 cSt, 6 cSt, or 8 cSt, respectively. Exemplary Heavy Base Oil may include a range of about 7 cSt to about 20 cSt. By way of further example, in one embodiment, Heavy Base Oil may include a range of about 7 cSt to about 17 cSt. By way of further example, in one embodiment, Heavy Base Oil may include a range of about 7 cSt to about 12 cSt. By way of further example, in one embodiment, Heavy Base Oil may include a range of about 7 cSt to about 12 cSt. By way of further example, in one embodiment, Heavy Base Oil may include a range of about 7 cSt to about 9 cSt. By way of further example, in one embodiment, Heavy Base Oil may include 7 cSt, 9 cSt, 12 cSt, 17 cSt, or 20 cSt, respectively.

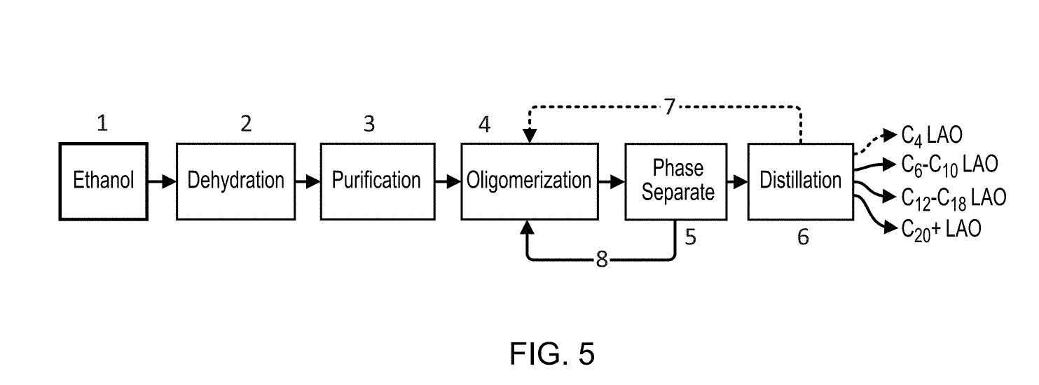

In general, the present disclosure further provides a process for the generation of LAOs from ethanol. Referring now to FIG. 5, ethanol feedstock 1 may be characterized by having ≥95% vol ethanol, <100 ppm wt of acetaldehyde (and even <250 ppm acetaldehyde), no more than 50 mg/L acids, such as about 10 mg/mL acids, no more than 0.3 vol % methanol, such as about 0.3% methanol, and no more than 1 ppm by wt of sulfur compounds, such as about 0.5 ppm wt of elemental sulfur. Ethanol feedstock 1 is then dehydrated 2 to provide ethylene. Purification 3 may be characterized by selectivity parameters when conversion is about 99%. Exemplary selectivity parameters include ethylene composition, ethane composition, propylene composition, butylenes composition, and acetaldehyde composition. In one such exemplary embodiment, the ethylene composition may be about 96.5, and even at least 96.5% ethylene monomer, the ethane composition may be about 0.5, and even no more than about 0.5 vol %, the propylene composition may be about 0.06, and even no more than about 0.06 vol %, the butylenes composition may be about 2.4, and even no more than about 2.4 vol %, and the acetaldehyde composition may be about <0.3. Following purification 3, the ethylene is subjected to oligomerization 4, phase separation 5, and distillation 6 to provide product LAOs. Optionally, unreacted ethylene may be recycled 8 back to oligomerization 4. Similarly, unreacted olefinic monomer(s) may also be recycled 7 back into oligomerization 4. Product LAOs include C4, C6-C10, C12-C18, and C20+ LAOs. Exemplary C4 LAOs include 1-butene. Exemplary C6-C10 LAOs include 1-hexene, 1-octene, 1-decene, and combinations thereof. Exemplary C12-C18 LAOs include 1-dodecene, 1-tetradecene, 1-hexadecene, 1-octadecene, and combinations thereof.

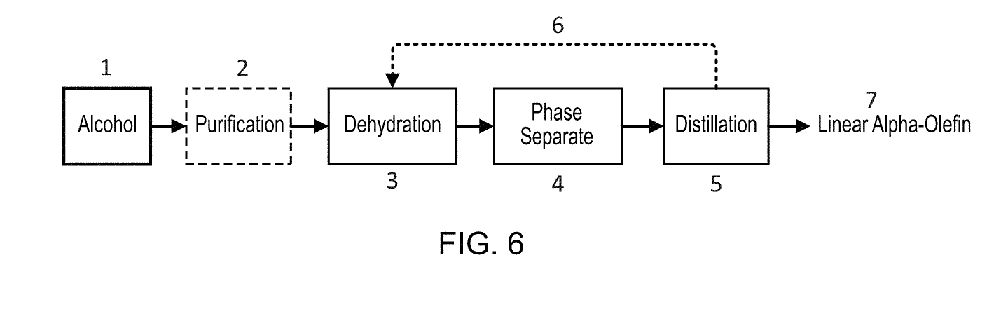

In general, the present disclosure further provides a process for the generation of LAOs from long-chain alcohols. Referring now to FIG. 6, exemplary long-chain alcohols 1 may include n-butanol, 1-pentanol, 1-hexanol, 1-heptanol, 1-octanol, 1-nonanol, 1-decanol, undecanol, dodecanol, tridecan-1-ol, 1-tetradecanol, 1-pentadecanol, cetyl alcohol, isobutanol, isoamyl alcohol, 2-methyl-1-butano, phenylethyl alcohol, tryptophol, and combinations thereof. Long-chain alcohols 1 are optionally subjected to purification 2, followed by dehydration 3, phase separation 4, and distillation 5 thereby providing LAOs 7. Optional recycling 6 of the unreacted feed (e.g., long-chain alcohols 1) back to dehydration 3 may improve yields of LAOs 7.

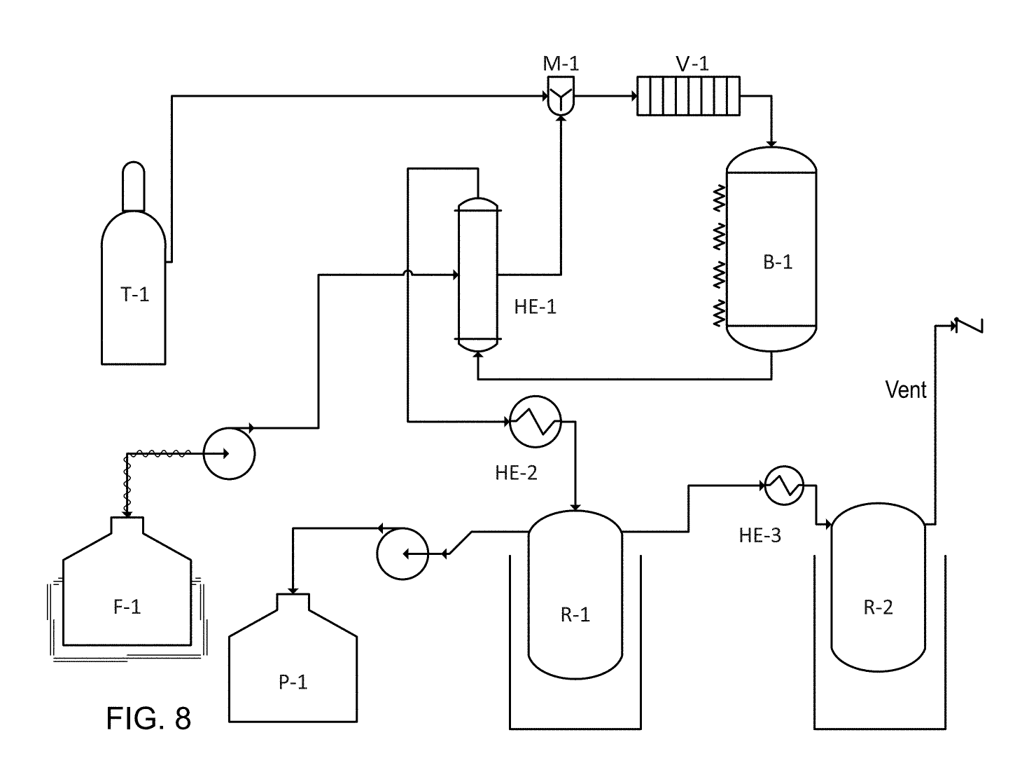

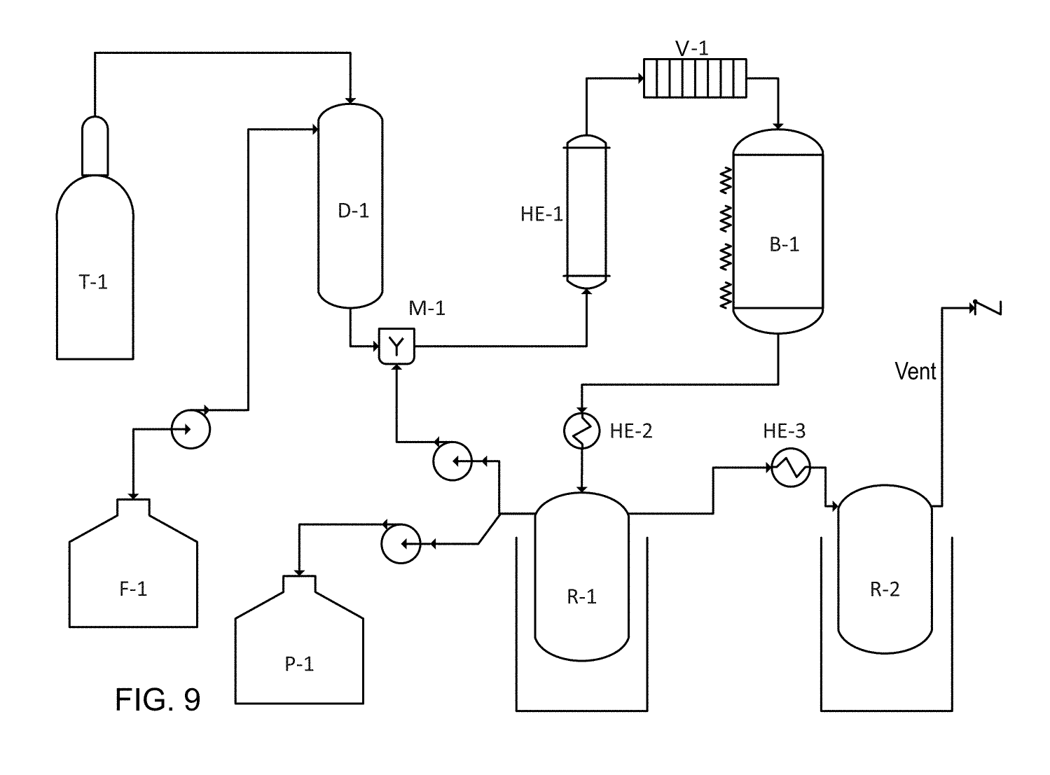

In general, embodiments of the present disclosure further provide a plurality of pilot dehydration reactor trains. Referring now to FIGS. 8 and 9, pilot dehydration reactor trains include a nitrogen feed tank T-1, a vessel F-1, a reactor B-1, a heat exchanger HE-1, HE-2 and HE-3, gas/liquid separator vessel R-1, gas trap vessel R-2, a product receiver P-1, and a vent. In general, inert gas from T-1 is fed to the reactor B-1 to remove oxygen from the process. Alcohol is then fed while heated from vessel F-1 to the reactor B-1 for dehydration. The dehydrated product is cooled by heat exchanger HE-1 and HE-2 and the condensed portion of the product is collected in vessel R-1. The uncondensed product is condensed in heat exchanger HE-3 and transferred to vessel R-2. The cooled product from R-1 is transferred to product receiver P-1, and this product, depending on the reaction conditions was purified olefins or a mixture of olefins, unreacted alcohol and byproducts (ethers and water). With respect to FIG. 9, a pilot reactor train includes a nitrogen gas tank T-1, a feed tank F-1, a drying bed (molecular sieves) purification vessel D-1, a vaporizer (electric heater) vessel V-1, a reactor (e.g., isothermal bed with a band heater or internal furnace) B-1, a heat exchanger HE-1, a heat exchanger HE-2, a vessel R-1, a vessel R-2, a final product tank, and a vent. Nitrogen or an inert gas T-1 was fed to the reactor train to remove oxygen from the process. Alcohol is fed from the feed tank to a molecular sieves purification vessel D-1. Then the purified feed was heated in heater vessel HE-1 and V-1 and fed to the reactor B-1 for dehydration. The dehydrated product was cooled via heat exchanger HE-2 and a condensed portion of the product was collected in vessel R-1. The uncondensed product was further cooled by heat exchanger HE-3 and collected in vessel R-2. The cooled liquid product in vessel R-1 was collected and depending on the reaction conditions was transferred to the final product tank or recycled back to heater vessel HE-1 for further dehydration in reactor B-1. In FIGS. 8-9: T-1 is an inert gas tank e.g. nitrogen; F-1 is a heated feed tank containing alcohol; D-1 is a drying bed e.g. Molecular Sieve; M-1 is a Mixer; V-1 is a vaporizer, B-1 is a dehydration reactor containing catalyst e.g. gamma alumina; HE-1 is a heat exchanger between hot vapor/liquid from B-1 and heated feed before a vaporizer; HE-2 is a heat exchanger to condense hot liquid before R-1; HE-3 is a heat exchanger before R-2; R-1 is a gas/liquid separator; R-2 is a gas trap before vent; and P-1 is dehydration product collection tank.

The oligomers of the present invention are characterized in that they are formed from several different monomer units, that can vary in carbon number, branch ratio, or reactive double bond position, chemically bonded into larger branched hydrocarbon molecules which comprise the hetero-oligomer reaction product(s), and form a statistical distribution which can be specified and measured. A hetero-oligomer is made of multiple different macromolecules (as opposed to a homo-oligomer that would be formed by a few identical molecules). In cases where the oligomers of the present invention are formed from several different monomer units, a percentage of the olefin monomers in the olefin monomer mixture may have a carbon number difference. For example, in one embodiment, at least 15% of the olefin monomers in the olefin monomer mixture may have a carbon number difference of at least four carbons. By way of further example, in one embodiment, at least 20% of the olefin monomers in the olefin monomer mixture may have a carbon number difference of at least four carbons. By way of further example, in one embodiment, at least 25% of the olefin monomers in the olefin monomer mixture may have a carbon number difference of at least four carbons. By way of further example, in one embodiment, at least 30% of the olefin monomers in the olefin monomer mixture may have a carbon number difference of at least four carbons. By way of further example, in one embodiment, at least 35% of the olefin monomers in the olefin monomer mixture may have a carbon number difference of at least four carbons. By way of further example, in one embodiment, at least 40% of the olefin monomers in the olefin monomer mixture may have a carbon number difference of at least four carbons. By way of further example, in one embodiment, at least 45% of the olefin monomers in the olefin monomer mixture may have a carbon number difference of at least four carbons. By way of further example, in one embodiment, at least 50% of the olefin monomers in the olefin monomer mixture may have a carbon number difference of at least four carbons. By way of further example, in one embodiment, at least 55% of the olefin monomers in the olefin monomer mixture may have a carbon number difference of at least four carbons. By way of further example, in one embodiment, at least 60% of the olefin monomers in the olefin monomer mixture may have a carbon number difference of at least four carbons. By way of further example, in one embodiment, at least 65% of the olefin monomers in the olefin monomer mixture may have a carbon number difference of at least four carbons. By way of further example, in one embodiment, at least 70% of the olefin monomers in the olefin monomer mixture may have a carbon number difference of at least four carbons. By way of further example, in one embodiment, at least 75% of the olefin monomers in the olefin monomer mixture may have a carbon number difference of at least four carbons. By way of further example, in one embodiment, at least 80% of the olefin monomers in the olefin monomer mixture may have a carbon number difference of at least four carbons.

In another embodiment, for example, at least 15% of the olefin monomers in the olefin monomer mixture may have a carbon number difference of at least five carbons. By way of further example, in one embodiment, at least 20% of the olefin monomers in the olefin mixture may have a carbon number difference of at least five carbons. By way of further example, in one embodiment, at least 25% of the olefin monomers in the olefin mixture may have a carbon number difference of at least five carbons. By way of further example, in one embodiment, at least 30% of the olefin monomers in the olefin mixture may have a carbon number difference of at least five carbons. By way of further example, in one embodiment, at least 35% of the olefin monomers in the olefin mixture may have a carbon number difference of at least five carbons. By way of further example, in one embodiment, at least 40% of the olefin monomers in the olefin mixture may have a carbon number difference of at least five carbons. By way of further example, in one embodiment, at least 45% of the olefin monomers in the olefin mixture may have a carbon number difference of at least five carbons. By way of further example, in one embodiment, at least 50% of the olefin monomers in the olefin mixture may have a carbon number difference of at least five carbons. By way of further example, in one embodiment, at least 55% of the olefin monomers in the olefin mixture may have a carbon number difference of at least five carbons. By way of further example, in one embodiment, at least 60% of the olefin monomers in the olefin mixture may have a carbon number difference of at least five carbons. By way of further example, in one embodiment, at least 65% of the olefin monomers in the olefin mixture may have a carbon number difference of at least five carbons. By way of further example, in one embodiment, at least 70% of the olefin monomers in the olefin mixture may have a carbon number difference of at least five carbons. By way of further example, in one embodiment, at least 75% of the olefin monomers in the olefin mixture may have a carbon number difference of at least five carbons. By way of further example, in one embodiment, at least 80% of the olefin monomers in the olefin mixture may have a carbon number difference of at least five carbons.

In yet another embodiment, for example, at least 15% of the olefin monomers in the olefin monomer mixture may have a carbon number difference of at least six carbons. By way of further example, in one embodiment, at least 20% of the olefin monomers in the olefin mixture may have a carbon number difference of at least six carbons. By way of further example, in one embodiment, at least 25% of the olefin monomers in the olefin mixture may have a carbon number difference of at least six carbons. By way of further example, in one embodiment, at least 30% of the olefin monomers in the olefin mixture may have a carbon number difference of at least six carbons. By way of further example, in one embodiment, at least 35% of the olefin monomers in the olefin mixture may have a carbon number difference of at least six carbons. By way of further example, in one embodiment, at least 40% of the olefin monomers in the olefin mixture may have a carbon number difference of at least six carbons. By way of further example, in one embodiment, at least 45% of the olefin monomers in the olefin mixture may have a carbon number difference of at least six carbons. By way of further example, in one embodiment, at least 50% of the olefin monomers in the olefin mixture may have a carbon number difference of at least six carbons. By way of further example, in one embodiment, at least 55% of the olefin monomers in the olefin mixture may have a carbon number difference of at least six carbons. By way of further example, in one embodiment, at least 60% of the olefin monomers in the olefin mixture may have a carbon number difference of at least six carbons. By way of further example, in one embodiment, at least 65% of the olefin monomers in the olefin mixture may have a carbon number difference of at least six carbons. By way of further example, in one embodiment, at least 70% of the olefin monomers in the olefin mixture may have a carbon number difference of at least six carbons. By way of further example, in one embodiment, at least 75% of the olefin monomers in the olefin mixture may have a carbon number difference of at least six carbons. By way of further example, in one embodiment, at least 80% of the olefin monomers in the olefin mixture may have a carbon number difference of at least six carbons.

In cases where the oligomers of the present invention are formed from several different monomer units, a percentage of the olefin monomers in the olefin monomer mixture may have a reactive double bond (olefinic) position. In certain embodiments, the reactive olefinic position may be an internal olefin bond or an external olefin bond. More specifically, a percentage of the olefin monomers in the olefin monomer mixture may have a reactive external olefinic bond, and further include an internal (i.e., non-reactive) olefinic bond. For example, in one embodiment, at least 0.1% of the olefin monomers in the olefin monomer mixture have an internal olefin bond. By way of further example, in one embodiment, at least 0.25% of the olefin monomers in the olefin monomer mixture have an internal olefin bond. By way of further example, in one embodiment, at least 0.5% of the olefin monomers in the olefin monomer mixture have an internal olefin bond. By way of further example, in one embodiment, at least 0.75% of the olefin monomers in the olefin monomer mixture have an internal olefin bond. By way of further example, in one embodiment, at least 1% of the olefin monomers in the olefin monomer mixture have an internal olefin bond. By way of further example, in one embodiment, at least 1.5% of the olefin monomers in the olefin monomer mixture have an internal olefin bond. By way of further example, in one embodiment, at least 1.75% of the olefin monomers in the olefin monomer mixture have an internal olefin bond. By way of further example, in one embodiment, at least 2% of the olefin monomers in the olefin monomer mixture have an internal olefin bond. By way of further example, in one embodiment, at least 3% of the olefin monomers in the olefin monomer mixture have an internal olefin bond. By way of further example, in one embodiment, at least 4% of the olefin monomers in the olefin monomer mixture have an internal olefin bond. By way of further example, in one embodiment, at least 5% of the olefin monomers in the olefin monomer mixture have an internal olefin bond.

In certain embodiments, no more than a percentage of the olefin monomers in the olefin monomer mixture include an internal olefin bond. For example, in one embodiment, no more than 4% of the olefin monomers in the olefin monomer mixture have an internal olefin bond. By way of further example, in one embodiment, no more than 3% of the olefin monomers in the olefin monomer mixture have an internal olefin bond. By way of further example, in one embodiment, no more than 2% of the olefin monomers in the olefin monomer mixture have an internal olefin bond. By way of further example, in one embodiment, no more than 1% of the olefin monomers in the olefin monomer mixture have an internal olefin bond.

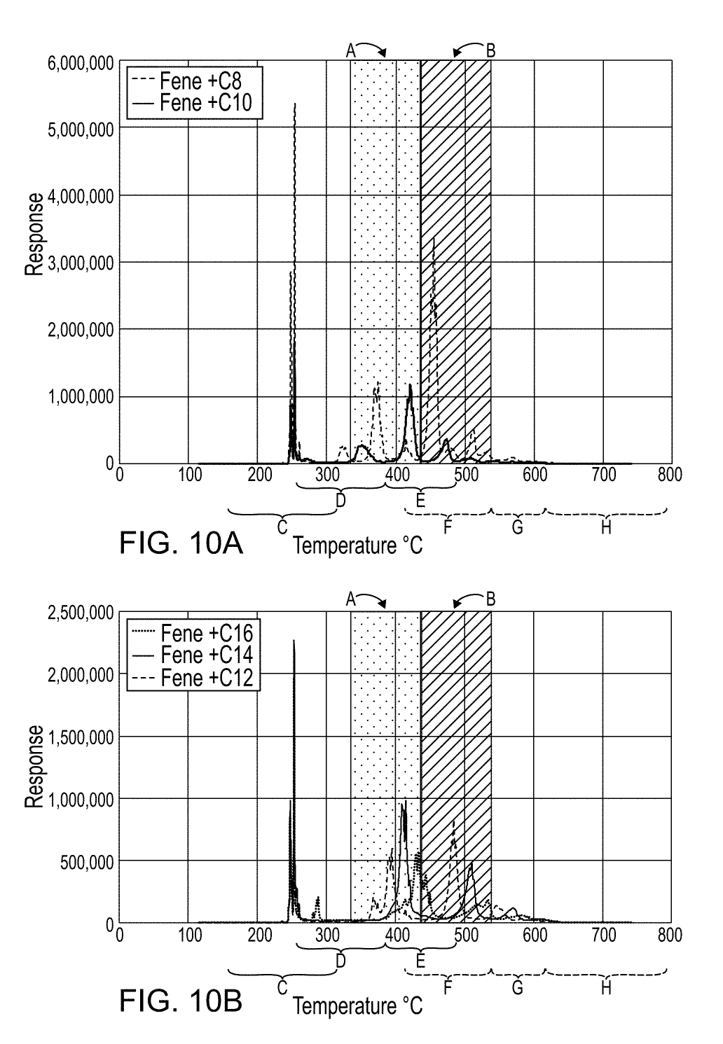

The boiling points, carbon numbers, and the molecular weights of the hetero-oligomers are correlated and exist as characteristic distributions which can be described as having some average values and more than one mode for each hetero-oligomer of a given order, such as dimer, trimer, tetramer etc. The modes of the distribution can be defined by considering the distribution along some axis such as molecular weight, carbon number, or actual or simulated boiling point as in FIG. 10A and FIG. 10B. When a distribution has multiple local maxima, as in the present case, it is common to refer to all the local maxima as modes of the distribution. Such a continuous distribution of oligomers is called multi-modal or polymodal (as opposed to unimodal). According to one embodiment, an oligomer product comprises a polymodal distribution of dimers, trimers and higher oligomers, where the dimer and trimer portions of the product have two or more distinct boiling point distributions which are separable by GC (Simdist) or physical separation by fractional, short-path or molecular distillation.

An advantage of the current invention can be seen when one considers that the physical properties of the hetero-oligomers vary continuously and significantly throughout the distribution and the spacing of the modes facilitates the physical separation of the oligomer product by fractional distillation into separate products with properties that can be controlled. In fact the properties of the final products can be more easily controlled and optimized than in the prior art by the careful selection of A) the monomer characteristics as mentioned; B) the relative amounts of each monomer which are incorporated in the oligomers; C) the reaction conditions which can alter selectivity of the reaction and the distribution of oligomers present in the reaction product; and D) the number and efficiency of the fractional separation stages. In one embodiment, fractional distillation is performed to separate the dimer portion of the branched saturated hydrocarbons into two or more product streams differing in boiling point or viscosity. In another embodiment, fractional distillation is performed to separate the trimer portion of the branched saturated hydrocarbons into two or more product streams differing in boiling point or viscosity. In yet another embodiment, fractional distillation is performed to separate the dimer and trimer portions of the branched saturated hydrocarbons into two or more product streams to adjust the Noack volatility, viscosity index and/or pour point of the branched saturated hydrocarbon product. In one embodiment, the branched saturated hydrocarbon mixture has a viscosity of less than 5 centistokes at 100 C, a viscosity index greater than 130 and a cold crank simulation (CCS) of less than 2100 at −35° C.

FIG. 11 shows an embodiment of a prior art distillation. In one embodiment of the prior art, the un-reacted alphaolefin and dimers of said alphaolefin are distilled off using a fractional distillation column. In a subsequent step the bottom products is further fractionated into a dimer cut (D1) and trimer cut (D2) and a bottoms product, predominantly trimer and tetramer, which according to one embodiment is no more than 10 cSt, also using a fractional distillation column.

FIG. 12 shows an embodiment of a C8-C16 distillation related to the inventive subject matter disclosed herein. According to one embodiment, oligomer product is passed to a distillation column to remove and/or recycle the unreacted olefin monomer (D1) and the bottoms (R1) are passed to a 2^{nd}, 3^{rd}, and 4^{th} distillation stage which can each be a fractional distillation column or alternatively a short-path evaporator. In a second stage a predominately dimer cut (D2) is taken overhead, typically 2-4 cSt and in the third and 4^{th} stage an early dimer and predominately trimer product is taken overhead (D3 and D4). In one embodiment D3 is up to 4 cSt and D4 is typically 5 cSt or more, and R4 can be between 20 and 20 cSt.

In one embodiment, base oils prepared as described herein are biodegradable. Biodegradability can be determined using one or more standardized test procedures and can provide valuable insight in comparing the potential risk of different lubricant products to the environment. One such guideline and test method has been set by the Organization for Economic Cooperation and Development (OECD) for degradation and accumulation testing.

The OECD has indicated that several tests may be used to determine the “ready biodegradability” of organic chemicals. Among these, aerobic ready biodegradability by the OECD 301B method tests material over a 28-day period and determines biodegradation of the material by measuring the evolution of carbon dioxide from the microbial oxidation of the material's organic carbon. The carbon dioxide produced is trapped in barium hydroxide solution and is quantified by titration of residual hydroxide with standardized hydrogen chloride. To determine the percent biodegradation, the amount of carbon dioxide (CO₂) produced microbially from the test material is compared to its theoretical carbon dioxide content (i.e., the complete oxidation of the carbon in the test material to CO₂). Positive controls, using sodium benzoate as a reference material, are run to check the viability of the aerobic microorganisms used in the procedure. Blank controls are also run in parallel. Tests, controls, and blanks are run in duplicate. In one embodiment, branched saturated hydrocarbons in a purified oligomer product have a biodegradability at 28 days as measured in accordance with OECD method 301b of at least 50%. In another embodiment, the branched saturated hydrocarbons may have a biodegradability at 28 days as measured in accordance with OECD method 301b of at least 60%. In another embodiment, the branched saturated hydrocarbons may have a biodegradability at 28 days as measured in accordance with OECD method 301b of at least 70%. In yet another embodiment, the branched saturated hydrocarbons may have a biodegradability at 28 days as measured in accordance with OECD method 301b of at least 75%. In yet a further embodiment, the branched saturated hydrocarbons have a biodegradability at 28 days as measured in accordance with OECD method 301b of at least 80%. In yet another embodiment, the branched saturated hydrocarbons may have a final (ultimate) biodegradability as measured in accordance with OECD method 301b of at least 60%. In yet another embodiment, the branched saturated hydrocarbons have a final (ultimate) biodegradability as measured in accordance with OECD method 301b of at least 70%. In yet another embodiment, the branched saturated hydrocarbons may have a final (ultimate) biodegradability as measured in accordance with OECD method 301b of at least 75%. In yet another embodiment, the branched saturated hydrocarbons may have a final (ultimate) biodegradability as measured in accordance with OECD method 301b of at least 80%. In yet another embodiment, the branched saturated hydrocarbons may have a final (ultimate) as measured in accordance with OECD 301b of at least method 88%. In yet another embodiment, the branched saturated hydrocarbons may have a final (ultimate) biodegradability as measured in accordance with OECD method 301b of at least 90%.

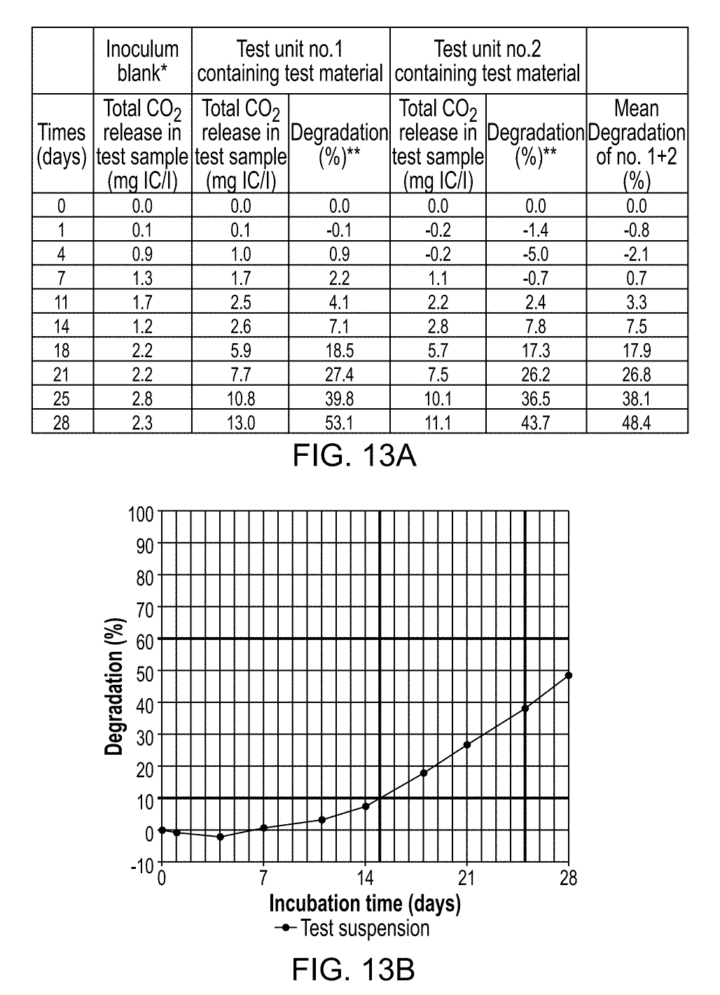

In FIG. 13A, an embodiment is shown of a prior art 28-day biodegradability study using the OECD 301b method for a commercial 4 cSt PAO. The study shows a mean 48.6% degradation in 28 days

In FIG. 13B, an embodiment is shown of a plot characterizing a 4 cSt commercial PAO base oil degradation in 28 days.

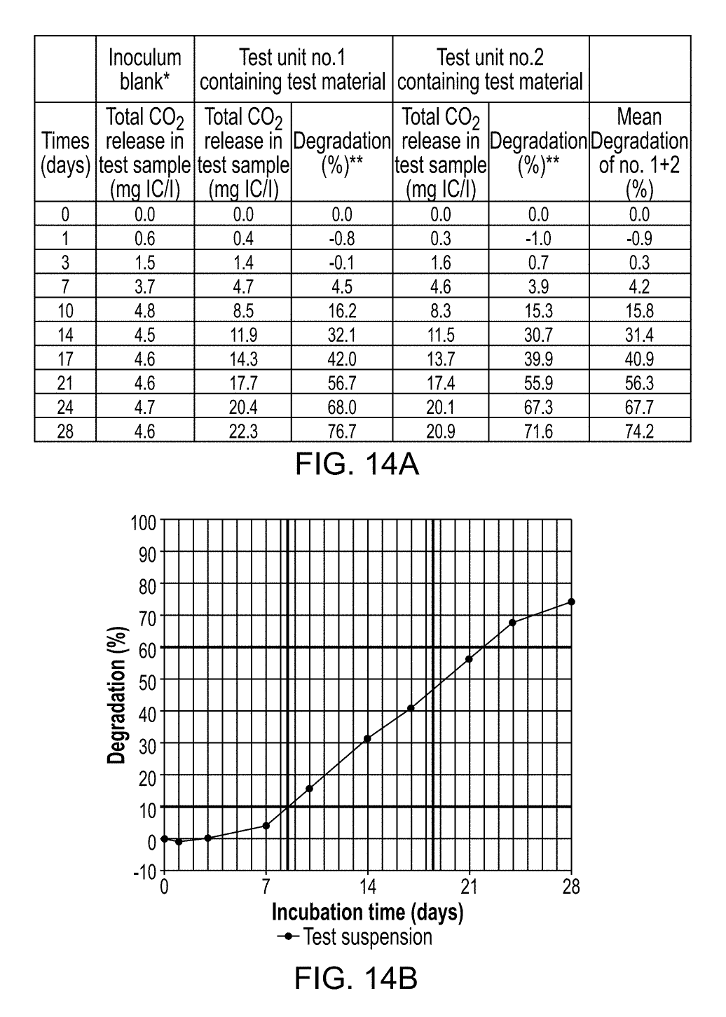

In FIG. 14A, an embodiment is shown of a 28-day biodegradability study related to the inventive subject matter disclosed herein using the OECD 301b method. The study shows a mean 74.2% degradability in 28 days.

In FIG. 14B, an embodiment is shown of a plot characterizing 4 cSt hydrocarbon base oil (e.g, using 50% LAO and 50% terpene co-monomers) related to the inventive subject matter disclosed herein.

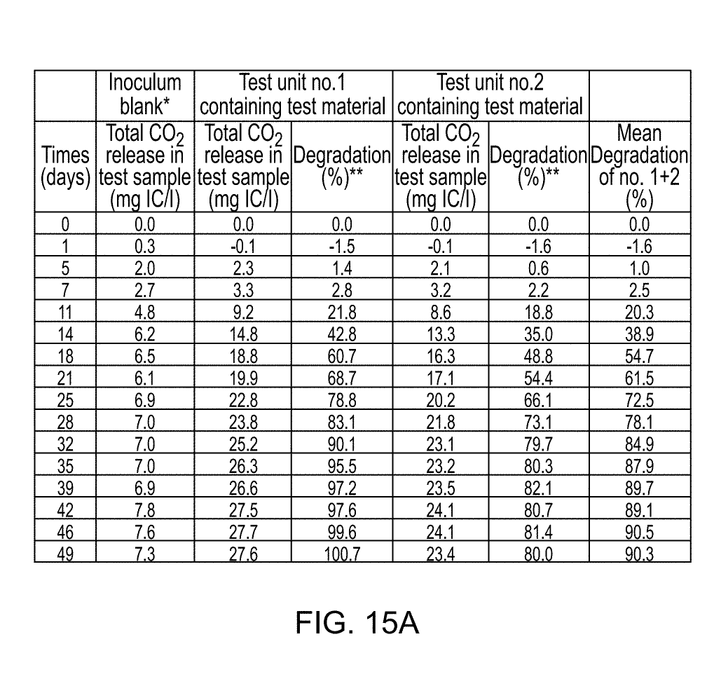

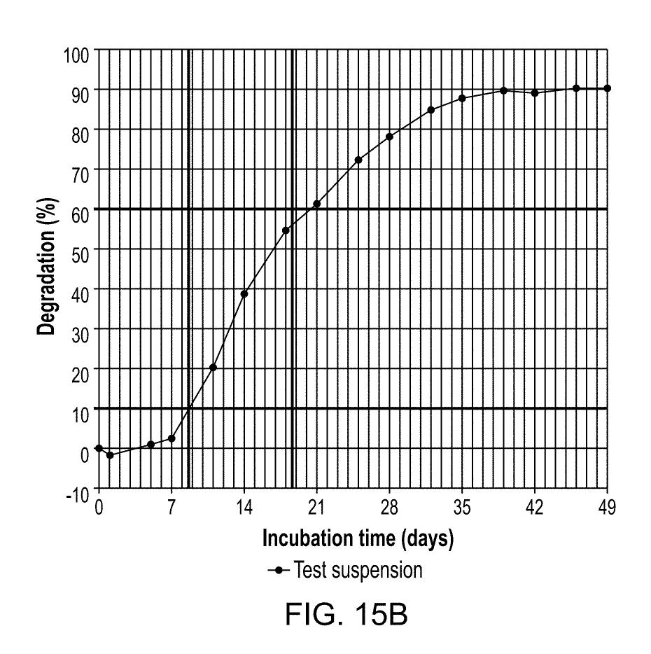

In FIG. 15A, an embodiment is shown of a 28-day and a 49-day biodegradability study related to the inventive subject matter disclosed herein using the OECD 301b method for a commercial 4 cSt PAO. The study shows a mean 90.3% degradation in 28 days.

In FIG. 15B, an embodiment is shown of a plot characterizing 5 cSt hydrocarbon base oil (e.g., using 50% LAO and 50% terpene co-monomers) related to the inventive subject matter disclosed herein.

As various changes could be made in the above articles, compositions and methods without departing from the scope of the disclosure, it is intended that all matter contained in the above description and shown in the accompanying drawings shall be interpreted as illustrative and not in a limiting sense.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

All directional descriptors, such as top, bottom, left, right, etc., are used solely for ease of reference with respect to the drawings and are not meant as limitations.

What is claimed is:

1. A process for the preparation of a mixture of branched saturated hydrocarbons, the process comprising:
(a) forming an oligomerization reaction mixture comprising an oligomerization catalyst system and an olefin monomer mixture, wherein the olefin monomer mixture has an average carbon number in the range of 9.5 to 13, and at least 10% of the olefin monomers in the olefin monomer mixture have a carbon number difference of at least four carbons,
(b) oligomerizing the olefin monomers in the oligomerization reaction mixture to produce an oligomer product comprising dimers, trimers, and higher oligomers,
(c) separating unreacted olefin monomer from the oligomer product to produce a purified oligomer product,
(d) hydrogenating the purified oligomer product to form a mixture of branched saturated hydrocarbons having a Bromine Index below 1000 as determined in accordance with ASTM D2710-09, and
(e) distilling the hydrogenated oligomer product.

2. The process of claim 1 wherein the olefin mixture has an average carbon number of 9.5 to 10.5.

3. The process of claim 1 wherein the olefin mixture has an average carbon number of 9.9 to 10.5.

4. The process of claim 1 wherein the olefin mixture has an average carbon number of 10.6 to 13.

5. The process of any preceding claim wherein the during the hydrogenation step, the oligomer product is isomerized.

6. The process of any preceding claim wherein at least 15% of the olefin monomers in the olefin monomer mixture have a carbon number difference of at least four carbons.

7. The process of any preceding claim wherein at least 20% of the olefin monomers in the olefin monomer mixture have a carbon number difference of at least four carbons.

8. The process of any preceding claim wherein at least 25% of the olefin monomers in the olefin monomer mixture have a carbon number difference of at least four carbons.

9. The process of any preceding claim wherein at least 30% of the olefin monomers in the olefin monomer mixture have a carbon number difference of at least four carbons.

10. The process of any preceding claim wherein at least 35% of the olefin monomers in the olefin monomer mixture have a carbon number difference of at least four carbons.

11. The process of any preceding claim wherein at least 40% of the olefin monomers in the olefin monomer mixture have a carbon number difference of at least four carbons.

12. The process of any preceding claim wherein at least 45% of the olefin monomers in the olefin monomer mixture have a carbon number difference of at least four carbons.

13. The process of any preceding claim wherein at least 50% of the olefin monomers in the olefin monomer mixture have a carbon number difference of at least four carbons.

14. The process of any preceding claim wherein at least 55% of the olefin monomers in the olefin monomer mixture have a carbon number difference of at least four carbons.

15. The process of any preceding claim wherein at least 60% of the olefin monomers in the olefin monomer mixture have a carbon number difference of at least four carbons.

16. The process of any preceding claim wherein at least 65% of the olefin monomers in the olefin monomer mixture have a carbon number difference of at least four carbons.

17. The process of any preceding claim wherein at least 70% of the olefin monomers in the olefin monomer mixture have a carbon number difference of at least four carbons.

18. The process of any preceding claim wherein at least 75% of the olefin monomers in the olefin monomer mixture have a carbon number difference of at least four carbons.

19. The process of any preceding claim wherein at least 80% of the olefin monomers in the olefin monomer mixture have a carbon number difference of at least four carbons.

20. The process of any preceding claim wherein at least 15% of the olefin monomers in the olefin monomer mixture have a carbon number difference of at least five carbons.

21. The process of any preceding claim wherein at least 20% of the olefin monomers in the olefin monomer mixture have a carbon number difference of at least five carbons.

22. The process of any preceding claim wherein at least 25% of the olefin monomers in the olefin monomer mixture have a carbon number difference of at least five carbons.

23. The process of any preceding claim wherein at least 30% of the olefin monomers in the olefin monomer mixture have a carbon number difference of at least five carbons.

24. The process of any preceding claim wherein at least 35% of the olefin monomers in the olefin monomer mixture have a carbon number difference of at least five carbons.

25. The process of any preceding claim wherein at least 40% of the olefin monomers in the olefin monomer mixture have a carbon number difference of at least five carbons.

26. The process of any preceding claim wherein at least 45% of the olefin monomers in the olefin monomer mixture have a carbon number difference of at least five carbons.

27. The process of any preceding claim wherein at least 50% of the olefin monomers in the olefin monomer mixture have a carbon number difference of at least five carbons.

28. The process of any preceding claim wherein at least 55% of the olefin monomers in the olefin monomer mixture have a carbon number difference of at least five carbons.

29. The process of any preceding claim wherein at least 60% of the olefin monomers in the olefin monomer mixture have a carbon number difference of at least five carbons.

30. The process of any preceding claim wherein at least 65% of the olefin monomers in the olefin monomer mixture have a carbon number difference of at least five carbons.

31. The process of any preceding claim wherein at least 70% of the olefin monomers in the olefin monomer mixture have a carbon number difference of at least five carbons.

32. The process of any preceding claim wherein at least 75% of the olefin monomers in the olefin monomer mixture have a carbon number difference of at least five carbons.

33. The process of any preceding claim wherein at least 80% of the olefin monomers in the olefin monomer mixture have a carbon number difference of at least five carbons.

34. The process of any preceding claim wherein less than 15% of the olefin monomers in the olefin monomer mixture have a carbon number difference of at least six carbons.

35. The process of any preceding claim wherein less than 20% of the olefin monomers in the olefin monomer mixture have a carbon number difference of at least six carbons.

36. The process of any preceding claim wherein less than 25% of the olefin monomers in the olefin monomer mixture have a carbon number difference of at least six carbons.

37. The process of any preceding claim wherein less than 30% of the olefin monomers in the olefin monomer mixture have a carbon number difference of at least six carbons.

38. The process of any preceding claim wherein less than 35% of the olefin monomers in the olefin monomer mixture have a carbon number difference of at least six carbons.

39. The process of any preceding claim wherein less than 40% of the olefin monomers in the olefin monomer mixture have a carbon number difference of at least six carbons.

40. The process of any preceding claim wherein less than 45% of the olefin monomers in the olefin monomer mixture have a carbon number difference of at least six carbons.

41. The process of any preceding claim wherein less than 50% of the olefin monomers in the olefin monomer mixture have a carbon number difference of at least six carbons.

42. The process of any preceding claim wherein less than 55% of the olefin monomers in the olefin monomer mixture have a carbon number difference of at least six carbons.

43. The process of any preceding claim wherein less than 60% of the olefin monomers in the olefin monomer mixture have a carbon number difference of at least six carbons.

44. The process of any preceding claim wherein less than 65% of the olefin monomers in the olefin monomer mixture have a carbon number difference of at least six carbons.

45. The process of any preceding claim wherein less than 70% of the olefin monomers in the olefin monomer mixture have a carbon number difference of at least six carbons.

46. The process of any preceding claim wherein less than 75% of the olefin monomers in the olefin monomer mixture have a carbon number difference of at least six carbons.

47. The process of any preceding claim wherein less than 80% of the olefin monomers in the olefin monomer mixture have a carbon number difference of at least six carbons.

48. The process of any preceding claim wherein at least 0.1% of the olefin monomers in the olefin monomer mixture have an internal olefin bond (carbon-carbon double bond).

49. The process of any preceding claim wherein at least 0.25% of the olefin monomers in the olefin monomer mixture have an internal olefin bond (carbon-carbon double bond).

50. The process of any preceding claim wherein at least 0.5% of the olefin monomers in the olefin monomer mixture have an internal olefin bond (carbon-carbon double bond).

51. The process of any preceding claim wherein at least 0.75% of the olefin monomers in the olefin monomer mixture have an internal olefin bond (carbon-carbon double bond).

52. The process of any preceding claim wherein at least 1% of the olefin monomers in the olefin monomer mixture have an internal olefin bond (carbon-carbon double bond).

53. The process of any preceding claim wherein at least 1.5% of the olefin monomers in the olefin monomer mixture have an internal olefin bond (carbon-carbon double bond).

54. The process of any preceding claim wherein at least 1.75% of the olefin monomers in the olefin monomer mixture have an internal olefin bond (carbon-carbon double bond).

55. The process of any preceding claim wherein at least 2% of the olefin monomers in the olefin monomer mixture have an internal olefin bond (carbon-carbon double bond).

56. The process of any preceding claim wherein at least 3% of the olefin monomers in the olefin monomer mixture have an internal olefin bond (carbon-carbon double bond).

57. The process of any preceding claim wherein at least 4% of the olefin monomers in the olefin monomer mixture have an internal olefin bond (carbon-carbon double bond).

58. The process of any preceding claim wherein at least 5% of the olefin monomers in the olefin monomer mixture have an internal olefin bond (carbon-carbon double bond).

59. The process of any preceding claim wherein no more than 4% of the olefin monomers in the olefin monomer mixture have an internal olefin bond (carbon-carbon double bond).

60. The process of any preceding claim wherein no more than 3% of the olefin monomers in the olefin monomer mixture have an internal olefin bond (carbon-carbon double bond).

61. The process of any preceding claim wherein no more than 2% of the olefin monomers in the olefin monomer mixture have an internal olefin bond (carbon-carbon double bond).

62. The process of any preceding claim wherein no more than 1% of the olefin monomers in the olefin monomer mixture have an internal olefin bond (carbon-carbon double bond).

63. The process of any preceding claim wherein at least 10% of the carbons comprised by the olefin monomers is renewable or biobased carbon as determined in accordance with ASTM D6866-11.

64. The process of any preceding claim wherein at least 20% of the carbons comprised by the olefin monomers is renewable or biobased carbon as determined in accordance with ASTM D6866-11.

65. The process of any preceding claim wherein at least 30% of the carbons comprised by the olefin monomers is renewable or biobased carbon as determined in accordance with ASTM D6866-11.

66. The process of any preceding claim wherein at least 40% of the carbons comprised by the olefin monomers is renewable or biobased carbon as determined in accordance with ASTM D6866-11.

67. The process of any preceding claim wherein at least 50% of the carbons comprised by the olefin monomers is renewable or biobased carbon as determined in accordance with ASTM D6866-11.

68. The process of any preceding claim wherein at least 60% of the carbons comprised by the olefin monomers is renewable or biobased carbon as determined in accordance with ASTM D6866-11.

69. The process of any preceding claim wherein at least 70% of the carbons comprised by the olefin monomers is renewable or biobased carbon as determined in accordance with ASTM D6866-11.

70. The process of any preceding claim wherein at least 80% of the carbons comprised by the olefin monomers is renewable or biobased carbon as determined in accordance with ASTM D6866-11.

71. The process of any preceding claim wherein at least 90% of the carbons comprised by the olefin monomers is renewable or biobased carbon as determined in accordance with ASTM D6866-11.

72. The process of any preceding claim wherein at least 95% of the carbons comprised by the olefin monomers is renewable or biobased carbon as determined in accordance with ASTM D6866-11.

73. The process of any preceding claim wherein 100% of the carbons comprised by the olefin monomers is renewable or biobased carbon as determined in accordance with ASTM D6866-11.

74. The process of any preceding claim wherein 10%-90% of the carbons comprised by the olefin monomers is renewable or biobased carbon as determined in accordance with ASTM D6866-11.

75. The process of any preceding claim wherein the olefin monomer mixture comprises a first olefin monomer population derived from primary, secondary or tertiary alcohols.

76. The process of claim 75 wherein the process further comprises dehydrating the primary, secondary, or tertiary alcohol in the presence of a γ-alumina catalyst at a pressure in the range of 0.1 to 30 psia, and a temperature within the range of 260-350° C. to form the first olefin monomer population.

77. The process of claim 75 or 76 wherein the first olefin monomer population comprises primary and secondary alcohols.

78. The process of claim 75 or 76 wherein the first olefin monomer population comprises primary and tertiary alcohols.

79. The process of claim 75 or 76 wherein the first olefin monomer population comprises primary, secondary and tertiary alcohols.

80. The process of any of claims 75 to 79 wherein the primary, secondary and/or tertiary alcohol comprises renewable carbon as determined in accordance with ASTM D6866-11.

81. The process of any preceding claim wherein the olefin monomer mixture comprises an alkene lacking renewable carbon as determined in accordance with ASTM D6866-11.

82. The process of any preceding claim wherein the olefin monomer mixture comprises less than 20 wt % decene based upon the weight of the olefins in the olefin mixture.

83. The process of any preceding claim wherein the olefin monomer mixture comprises less than 15 wt % decene based upon the weight of the olefins in the olefin mixture.

84. The process of any preceding claim wherein the olefin monomer mixture comprises less than 10 wt % decene based upon the weight of the olefins in the olefin mixture.

85. The process of any preceding claim wherein the olefin monomer mixture comprises less than 5 wt % decene based upon the weight of the olefins in the olefin mixture.

86. The process of any preceding claim wherein the olefin monomer mixture comprises less than 1 wt % decene based upon the weight of the olefins in the olefin mixture.

87. The process of any preceding claim wherein the olefin monomer mixture comprises no decene.

88. The process of any of preceding claim wherein the olefin mixture comprises a terpene.

89. The process of any preceding claim wherein the olefin mixture comprises a terpene but less than 50 wt % terpene, based upon the weight of the olefins in the olefin mixture.

90. The process of any preceding claim wherein the olefin mixture comprises 5 to 50 wt % terpene, based upon the weight of the olefins in the olefin mixture.

91. The process of any preceding claim wherein the olefin mixture comprises at least one sesquiterpene.

92. The process of any preceding claim wherein the olefin mixture comprises at least one sesquiterpenes but less than 50 wt % sesquiterpene, based upon the weight of the olefins in the olefin mixture.

93. The process of any preceding claim wherein the olefin mixture comprises 5 to 50 wt % sesquiterpene, based upon the weight of the olefins in the olefin mixture.

94. The process of any preceding claim wherein the olefin mixture comprises 10 to 50 wt % sesquiterpene, based upon the weight of the olefins in the olefin mixture.

95. The process of any preceding claim wherein the olefin mixture comprises 15 to 50 wt % sesquiterpene, based upon the weight of the olefins in the olefin mixture.

96. The process of any preceding claim wherein the olefin mixture comprises 25 to 50 wt % sesquiterpene, based upon the weight of the olefins in the olefin mixture.

97. The process of any preceding claim wherein the olefin mixture comprises 10 to 40 wt % sesquiterpene, based upon the weight of the olefins in the olefin mixture.

98. The process of any preceding claim wherein the olefin mixture comprises 25 to 40 wt % sesquiterpene, based upon the weight of the olefins in the olefin mixture.

99. The process of any preceding claim wherein the olefin mixture comprises 10 to 25 wt % sesquiterpene, based upon the weight of the olefins in the olefin mixture.

100. The process of any preceding claim wherein the olefin mixture is treated to remove impurities prior to the oligomerization step.

101. The process of any preceding claim wherein oligomerizing the olefin monomers in the oligomerization reaction mixture produces an oligomer product comprising a polymodal distribution of dimers, trimers, and higher oligomers, where the dimer and trimer portions of the product have two or more distinct boiling point distributions which are separable by GC (Simdist) or physical separation by fractional, short-path, or molecular distillation.

102. The process of any preceding claim wherein the branched saturated hydrocarbons have a biodegradability at 28 days as measured in accordance with OECD method 301b of at least 50%.

103. The process of any preceding claim wherein the branched saturated hydrocarbons have a biodegradability at 28 days as measured in accordance with OECD method 301b of at least 60%.

104. The process of any preceding claim wherein the branched saturated hydrocarbons have a biodegradability at 28 days as measured in accordance with OECD method 301b of at least 70%.

105. The process of any preceding claim wherein the branched saturated hydrocarbons have a biodegradability at 28 days as measured in accordance with OECD method 301b of at least 75%.

106. The process of any preceding claim wherein the branched saturated hydrocarbons have a biodegradability at 28 days as measured in accordance with OECD method 301b of at least 80%.

107. The process of any preceding claim wherein the branched saturated hydrocarbons have a final (ultimate) biodegradability as measured in accordance with OECD method 301b of at least 60%.

108. The process of any preceding claim wherein the branched saturated hydrocarbons have a final (ultimate) biodegradability as measured in accordance with OECD method 301b of at least 70%.

109. The process of any preceding claim wherein the branched saturated hydrocarbons have a final (ultimate) biodegradability as measured in accordance with OECD method 301b of at least 75%.

110. The process of any preceding claim wherein the branched saturated hydrocarbons have a final (ultimate) biodegradability as measured in accordance with OECD method 301b of at least 80%.

111. The process of any preceding claim wherein the branched saturated hydrocarbons have a final (ultimate) as measured in accordance with OECD 301b of at least method 88%.

112. The process of any preceding claim wherein the branched saturated hydrocarbons have a final (ultimate) biodegradability as measured in accordance with OECD method 301b of at least 90%.

113. The process of any preceding claim wherein the process further comprises fractional distillation to separate the dimer portion of the branched saturated hydrocarbons into two or more product streams differing in boiling point or viscosity.

114. The process of any preceding claim wherein the process further comprises fractional distillation to separate the trimer portion of the branched saturated hydrocarbons into two or more product streams differing in boiling point or viscosity.

115. The process of any preceding claim wherein the process further comprises fractional distillation to separate the dimer and trimer portions of the branched saturated hydrocarbons into two or more product streams to adjust the Noack volatility, viscosity index and/or pour point of the branched saturated hydrocarbon product.

116. A process for the preparation of a linear alpha olefin mixture, the process comprising:
(a) forming a dehydration reaction mixture comprising a dehydration catalyst and ethanol, the dehydration reaction mixture comprising at least 95 vol % ethanol, no more than 250 ppm by wt acetaldehyde, no more than 50 mg/L acids, no more than 0.3 vol % methanol and no more than 1 ppm by wt sulfur compounds (as S),
(b) dehydrating the reaction mixture to form a dehydration reaction product, the dehydration reaction product containing at least 96.5 vol % ethylene monomer, no more than 0.5 vol % ethane, no more than 0.06 vol % propylene, no more than 2.4 vol % butylenes and less than 0.3 vol % acetaldehyde,
(c) forming an oligomerization reaction mixture comprising an oligomerization catalyst system and the dehydration reaction product,
(d) oligomerizing the ethylene monomer in the oligomerization reaction mixture to produce an oligomer product comprising a mixture of linear alpha olefins
(e) separating unreacted ethylene monomer from the oligomer product to form a purified oligomer product comprising a mixture of linear alpha olefins, and
(f) distilling the purified oligomer product into linear alpha olefin fractions, each of the different linear alpha olefins having different carbon numbers.

117. A process for the preparation of a linear alpha olefin mixture, the process comprising:
(a) forming a dehydration reaction mixture comprising a dehydration catalyst and ethanol, the dehydration reaction mixture comprising at least 95 vol % ethanol, no more than 100 ppm by wt acetaldehyde, no more than 50 mg/L acids, no more than 0.3 vol % methanol and no more than 1 ppm by wt sulfur compounds (as S),
(b) dehydrating the reaction mixture to form a dehydration reaction product, the dehydration reaction product containing at least 96.5 vol % ethylene monomer, no more than 0.5 vol % ethane, no more than 0.06 vol % propylene, no more than 2.4 vol % butylenes and less than 0.3 vol % acetaldehyde,
(c) forming an oligomerization reaction mixture comprising an oligomerization catalyst system and the dehydration reaction product,
(d) oligomerizing the ethylene monomer in the oligomerization reaction mixture to produce an oligomer product comprising a mixture of linear alpha olefins
(e) separating unreacted ethylene monomer from the oligomer product to form a purified oligomer product comprising a mixture of linear alpha olefins, and
(f) distilling the purified oligomer product into linear alpha olefin fractions, each of the different linear alpha olefins having different carbon numbers.

118. A process for the preparation of a linear alpha olefin mixture, the process comprising:
(a) forming a dehydration reaction mixture comprising a dehydration catalyst and ethanol, the dehydration reaction mixture comprising at least 95 vol % ethanol, no more than 100 ppm by wt acetaldehyde, no more than 50 mg/L acids, no more than 0.3 vol % methanol and no more than 1 ppm by wt sulfur compounds (as S),
(b) dehydrating the reaction mixture to form a dehydration reaction product, the dehydration reaction product containing at least 96.5 vol % ethylene monomer, no more than 0.5 vol % ethane, no more than 0.06 vol % propylene, no more than 2.4 vol % butylenes and less than 0.3 vol % acetaldehyde,
(c) forming an oligomerization reaction mixture comprising an oligomerization catalyst system and the dehydration reaction product,
(d) oligomerizing the ethylene monomer in the oligomerization reaction mixture to produce an oligomer product comprising a mixture of linear alpha olefins
(e) separating unreacted ethylene monomer from the oligomer product to form a purified oligomer product comprising a mixture of linear alpha olefins, and
(f) distilling the purified oligomer product into linear alpha olefin fractions, each of the different linear alpha olefins having different carbon numbers.

119. A process for the preparation of a mixture of branched saturated hydrocarbons, the process comprising:
(a) forming an oligomerization reaction mixture comprising an oligomerization catalyst system and an olefin monomer mixture, the olefin monomer mixture comprising 25-50 wt % octene, 15-50 wt % dodecene and less than 25 wt % decene based upon the weight of the olefins in the olefin mixture, the olefin mixture having an average carbon number in the range of 9.5 to 10.5,
(b) oligomerizing the olefin monomers in the oligomerization reaction mixture to produce an oligomer product comprising dimers, trimers, and higher oligomers,
(c) separating unreacted olefin monomer from the oligomer product to produce a purified oligomer product, and
(d) hydrogenating the purified oligomer product to form a mixture of branched saturated hydrocarbons having a Bromine Index below 1000 as determined in accordance with ASTM D2710-09.

120. The process of claim 119 wherein the olefin monomer mixture comprises less than 20 wt % decene based upon the weight of the olefins in the olefin mixture.

121. The process of claim 119 wherein the olefin monomer mixture comprises less than 15 wt % decene based upon the weight of the olefins in the olefin mixture.

122. The process of claim 119 wherein the olefin monomer mixture comprises less than 10 wt % decene based upon the weight of the olefins in the olefin mixture.

123. The process of claim 119 wherein the olefin monomer mixture comprises less than 5 wt % decene based upon the weight of the olefins in the olefin mixture.

124. The process of claim 119 wherein the olefin monomer mixture comprises less than 1 wt % decene based upon the weight of the olefins in the olefin mixture.

125. The process of claim 119 wherein the olefin monomer mixture comprises no decene.

126. The process of any of claims 119 to 125 wherein the octene contains renewable carbon.

127. The process of any of claims 119 to 125 wherein the dodecene contains renewable carbon.

128. The process of any of claims 119 to 125 wherein the decene contains renewable carbon.

129. The process of any of claims 119 to 125 wherein the octene and the dodecene contains renewable carbon.

130. The process of any of claims 119 to 125 wherein the octene, dodecene and dodecene contain renewable carbon.

131. The process of any of claims 119 to 129 wherein the olefin mixture comprises a terpene.

132. The process of any of claims 119 to 129 wherein the olefin mixture comprises a terpene but less than 50 wt % terpene, based upon the weight of the olefins in the olefin mixture.

133. The process of any of claims 119 to 129 wherein the olefin mixture comprises 5 to 50 wt % terpene, based upon the weight of the olefins in the olefin mixture.

134. The process of any of claims 119 to 129 wherein the olefin mixture comprises at least one sesquiterpene.

135. The process of any of claims 119 to 129 wherein the olefin mixture comprises at least one sesquiterpenes but less than 50 wt % sesquiterpene, based upon the weight of the olefins in the olefin mixture.

136. The process of any of claims 119 to 129 wherein the olefin mixture comprises 5 to 50 wt % sesquiterpene, based upon the weight of the olefins in the olefin mixture.

137. The process of any of claims 119 to 136 wherein at least 10% of the carbon comprised by the olefin mixture is renewable or biobased carbon as determined in accordance with ASTM D6866-11.

138. The process of any of claims 119 to 136 wherein at least 20% of the carbon comprised by the olefin mixture is renewable or biobased carbon as determined in accordance with ASTM D6866-11.

139. The process of any of claims 119 to 136 wherein at least 30% of the carbon comprised by the olefin mixture is renewable or biobased carbon as determined in accordance with ASTM D6866-11.

140. The process of any of claims 119 to 136 wherein at least 40% of the carbon comprised by the olefin mixture is renewable or biobased carbon as determined in accordance with ASTM D6866-11.

141. The process of any of claims 119 to 136 wherein at least 50% of the carbon comprised by the olefin mixture is renewable or biobased carbon as determined in accordance with ASTM D6866-11.

142. The process of any of claims 119 to 136 wherein at least 60% of the carbon comprised by the olefin mixture is renewable or biobased carbon as determined in accordance with ASTM D6866-11.

143. The process of any of claims 119 to 136 wherein at least 70% of the carbon comprised by the olefin mixture is renewable or biobased carbon as determined in accordance with ASTM D6866-11.

144. The process of any of claims 119 to 136 wherein at least 80% of the carbon comprised by the olefin mixture is renewable or biobased carbon as determined in accordance with ASTM D6866-11.

145. The process of any of claims 119 to 136 wherein at least 90% of the carbon comprised by the olefin mixture is renewable or biobased carbon as determined in accordance with ASTM D6866-11.

146. The process of any preceding claim wherein the branched saturated hydrocarbon mixture has a viscosity of less than 5 centistokes at 100 C, a viscosity index greater than 130 and a cold crank simulation (CCS) of less than 2100 at −35° C.
